(19) United States
(12) Patent Application Publication
(10) Pub. No.: US 2013/0116203 A1
(43) Pub. Date: May 9, 2013

(54) ISOTHIOCYNATES AND GLUCOSINOLATE COMPOUNDS AND ANTI-TUMOR COMPOSITIONS CONTAINING SAME

(71) Applicants: Scott R. Rajski, Madison, WI (US); Jared Rae Mays, Madison, WI (US)

(72) Inventors: Scott R. Rajski, Madison, WI (US); Jared Rae Mays, Madison, WI (US)

(21) Appl. No.: 13/290,571

(22) Filed: Nov. 7, 2011

(51) Int. Cl. A61K 31/7028; A61K 31/44; A61K 31/4152; A61K 31/357; A61K 31/341; A61K 31/13; C07H 15/00; C07D 213/53; C07D 231/48; C07D 317/46; C07D 307/02; C07C 331/20; C07C 331/28; A61P 35/00; C12N 5/02

(52) U.S. Cl. 514/24; 435/375; 514/357; 514/407; 514/466; 514/471; 514/638; 536/17.4; 536/17.6; 546/331; 548/367.7; 549/441; 549/493; 558/10; 558/13

(57) ABSTRACT

The present invention provides glucosinolate and isothiocyanate compounds and related methods for synthesizing these compounds and analogs. In certain embodiments, these glucosinolate and isothiocyanate compounds are useful and chemopreventive and or chemotherapeutic agents.

### CROSS-REFERENCE TO RELATED APPLICATIONS

Priority is hereby claimed to provisional application Ser. No. 60/807,409, filed 14 Jul. 2006, which is incorporated herein by reference.

### STATEMENT REGARDING FEDERAL FUNDING

This invention was made with United States government support awarded by the following agency: NIH Grant Nos: CA117519 and RR021086. The United States has certain rights in this invention.

### FIELD OF THE INVENTION

The present invention is directed to isothiocyanate and glucosinolate compounds, anti-neoplastic pharmaceutical compositions containing these compounds, and corresponding methods to inhibit the growth of tumors by administering the compounds or compositions to a subject in need of such treatment.

### BACKGROUND

Diets rich in fruits and vegetables are associated with a reduced risk of degenerative diseases, including cancer and cardiovascular disease (1-2). In particular, cruciferous vegetables such as broccoli, cauliflower, watercress, Brussels sprouts, and cabbage are associated with these beneficial effects. Studies have implicated glucosinolates, and their downstream catabolites, isothiocynates (ITC's) as a likely source of these effects (3). For example, the glucosinolate glucoraphanin (compound 2; systematic name 4-methylsulfinylbutyl glucosinolate) is converted in plants to sulforaphane (compound 1; systematic name 1-isothiocyanato-4-(methylsulfinyl) butane. See FIG. 1. Isothiocyanates are just one type of the many catabolic products of glucosinolates (4). Glucosinolates and ITC's have received significant attention in the past decade as potential chemopreventive and chemotherapeutic agents (5-6). For example, many ITCs have been shown to inhibit chemically-induced carcinogenesis through enhanced detoxification of reactive carcinogens via the induction of phase II drug-metabolizing enzymes such as glutathione-S-transferases, NAD(P)H:quinone reductase, epoxide hydrolase and UDP-glucuronosyl-transferases (7-11). ITC's also inhibit carcinogen activation by reducing expression levels of phase I drug-metabolizing enzymes and stimulating apoptosis of damaged cells (12-15). As one class of catabolites of glucosinolates, the ITC's (i.e., compounds having the structure S═C═N—R) are thought to be at least partially responsible for the reduced risk of degenerative diseases in humans associated with the consumption of vegetables.

Sulforaphane in particular is an ITC that has been implicated as both a chemopreventive and chemotherapeutic agent capable of inhibiting carcinogenesis Sulforaphane is especially abundant in broccoli and has attracted significant attention since its identification in 1992 (7).

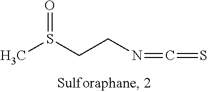

Accordingly, the need exists to explore isothiocyanate and glucosinolate compounds and related methods for use of these compounds as antitumor active and chemopreventive agents.

### SUMMARY OF THE INVENTION

The present invention relates to compositions of isothiocyanate and glucosinolate compounds and related methods for use of these compounds as antitumor active and chemopreventive agents. The invention is also directed to the use of these compounds to inhibit HDAC activity.

Thus, one version of the invention is direct to compounds of Formula I:

R—N═C═S

wherein R is selected from the group consisting of dimethylpropyl, C₃-C₁₀ mono- or bicycloalkyl, C₆-C₁₀ mono- or bicycloakenyl, halobenzyl, alkyloxybenzyl, tetrahydronaphthalenyl, biphenyl-C₁-C₆-alkyl, phenoxybenzyl-C₁-C₆-alkyl, and pyridinyl-C₁-C₆-alkyl, as well as N-acetyl cysteine conjugates thereof, and salts thereof. It is particularly preferred that R is selected from the group consisting of:

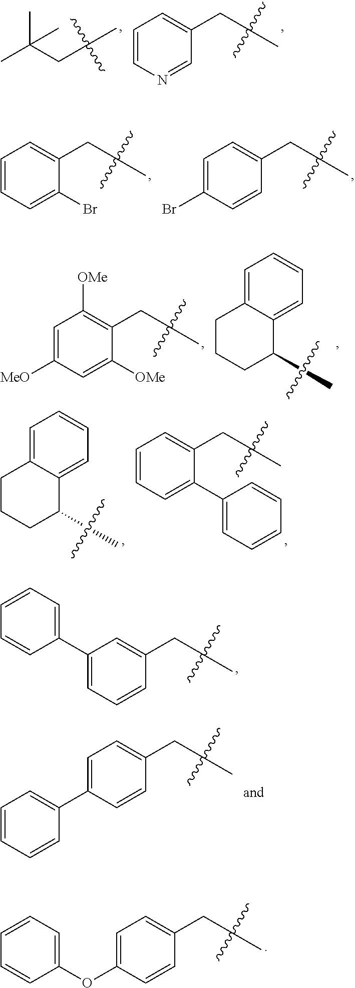

The invention is further directed to a pharmaceutical composition for inhibiting neoplastic cell growth comprising one or more compounds listed in the immediately preceding paragraph, or pharmaceutically suitable salts thereof, optionally in combination with a pharmaceutically-suitable carrier. The carrier may be any solid or liquid carrier now known in the art or developed in the future.

The invention is also directed to a method of inhibiting growth of cancer cells. The method comprises treating the cancer cells with an effective growth-inhibiting amount of one or more compounds described in the previous paragraphs, or pharmaceutically suitable salts thereof. The method includes administering to a human cancer patient (or other mammalian patient) in need thereof which is effective to inhibit the growth of the cancer. The compound(s) may be administered by any route now known in the art or developed in the future, including parenterally, intravenously, orally, etc.

Another version of the invention is directed to compounds of Formula II:

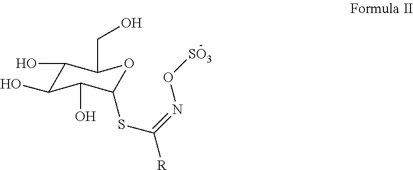

wherein R is selected from the group consisting of:

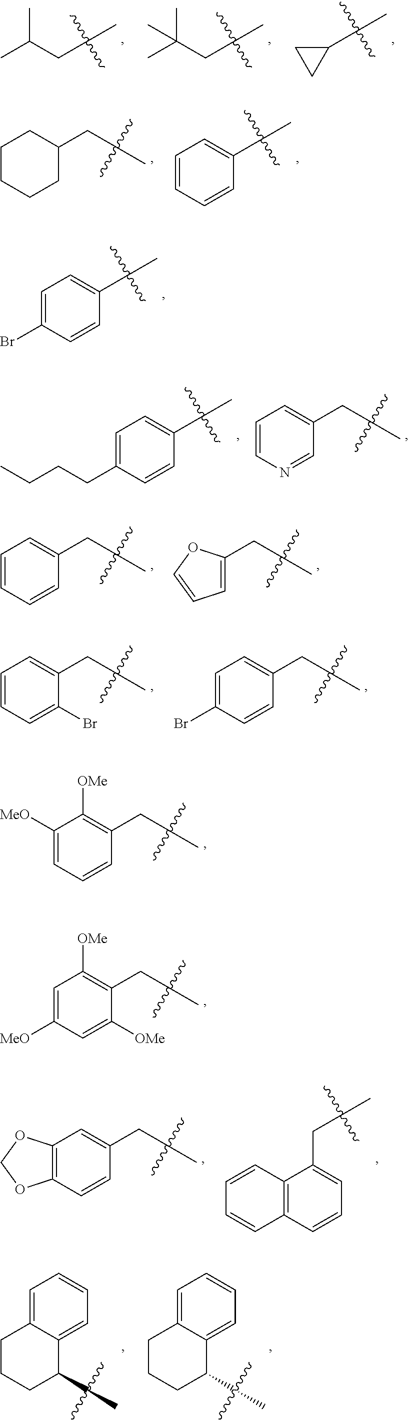

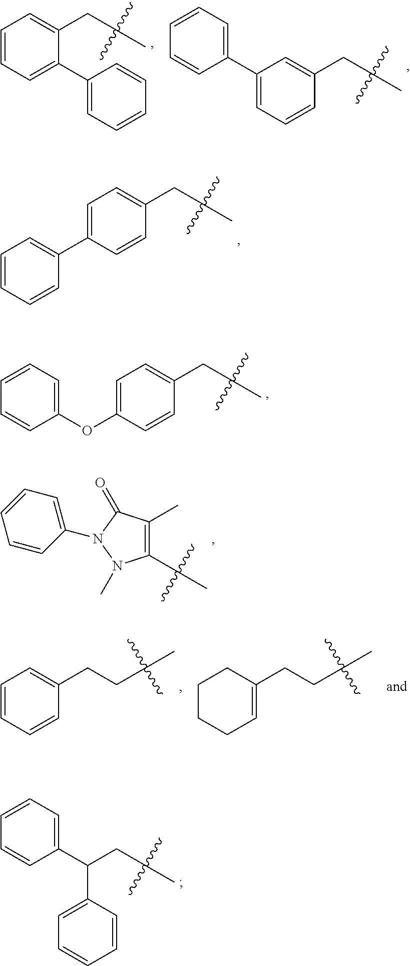

as well as N-acetyl cysteine conjugates thereof; and salts thereof.

These compounds may also be incorporated into a pharmaceutical composition for inhibiting neoplastic cell growth. Thus, the composition comprising one or more compounds recited in the immediately preceding paragraph, and/or a pharmaceutically suitable salts thereof, optionally in combination with a pharmaceutically-suitable carrier as described herein.

Likewise, the invention encompasses a method of inhibiting growth of cancer cells in mammals comprising administering to the mammal a cancer cell growth-inhibiting amount of one or more of compounds of Formula II as described above or a pharmaceutically suitable salt thereof. In this version of the invention, the amount of the administered Formula II compound yields an in vivo metabolite selected from the group consisting of:

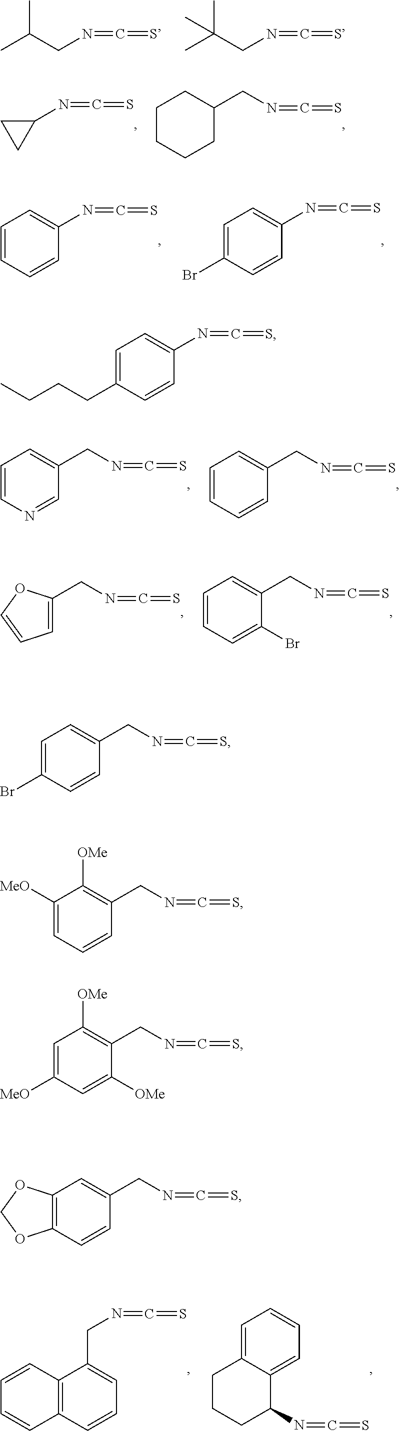

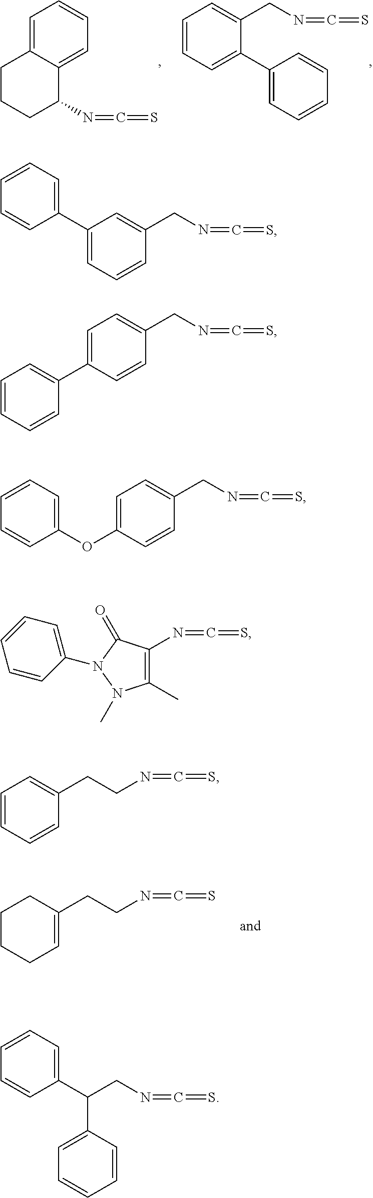

As in the prior methods, the amount of one or more of the compounds may be administered to a human cancer patient in need thereof (or other needful mammal) which is effective to inhibit the growth of the cancer. The compounds may be administered by any route now known in the art or developed in the future.

The invention also includes a pharmaceutical composition for inhibiting neoplastic cell growth comprising one or more isothiocyanate compounds recited in the preceding two paragraphs, as well as N-acetyl cysteine conjugates thereof, and pharmaceutically suitable salts thereof, and optionally in combination with a pharmaceutically-suitable carrier.

The invention also includes a method of inhibiting growth of cancer cells in mammals comprising administering to the mammal a cancer cell growth-inhibiting amount of one or more of compounds selected from the group consisting of:

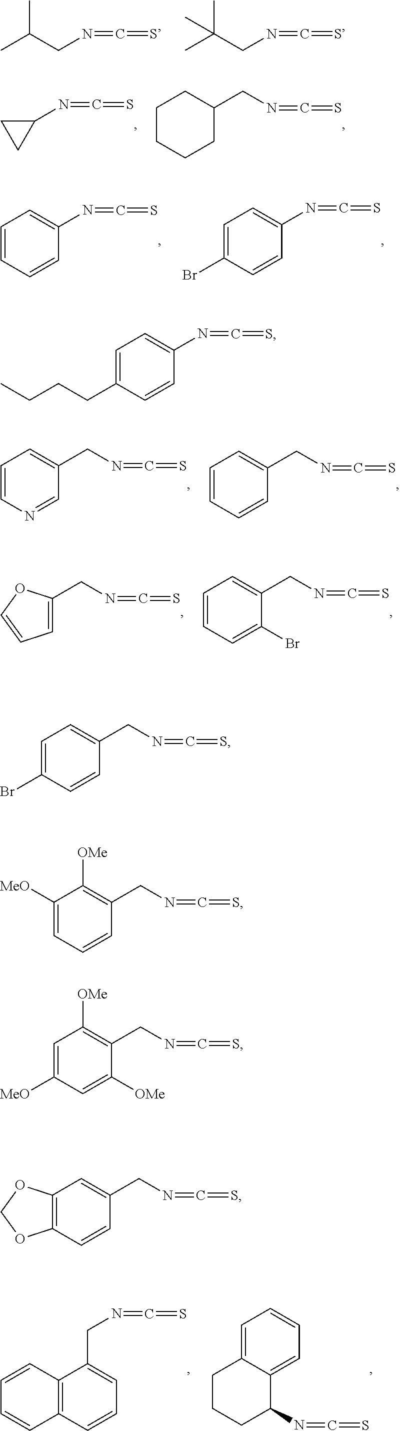

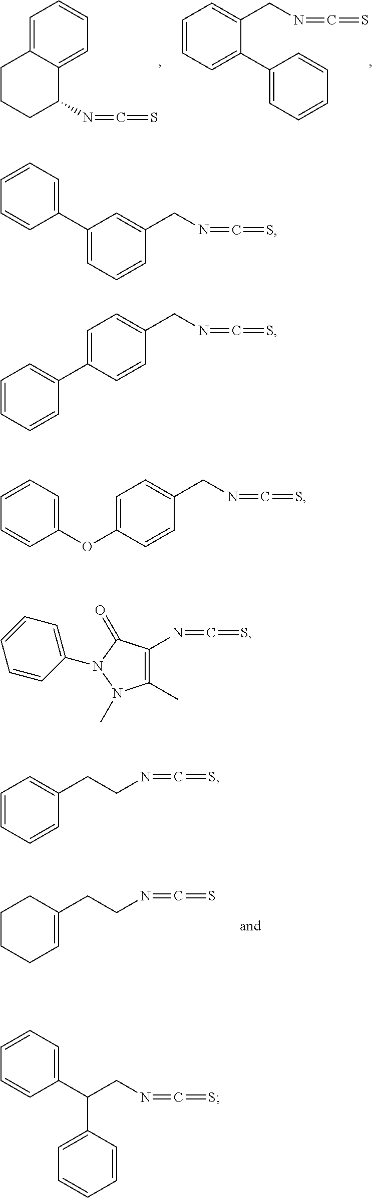

N-acetyl cysteine conjugates thereof;

and pharmaceutically suitable salts thereof.

Lastly, the invention includes a method of inhibiting histone deacetylase activity in mammals, including humans. The method comprises administering to the mammal a histone deacetylase activity-inhibiting amount of one or more of compounds of Formula II has described herein or a pharmaceutically suitable salt thereof.

For use in medicine, the salts of the compounds of Formulas (I) and (II) must be pharmaceutically suitable salts. Other salts may, however, be useful to make the compounds themselves, as well as their pharmaceutically acceptable salts. Pharmaceutically suitable salts of the compounds include all salts conventionally used in formulating pharmacologically active agents, including (without limitation) acid addition salts which may, for example, be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid. Furthermore, where the compounds of the invention carry an acidic moiety, suitable pharmaceutically acceptable salts thereof may include alkali metal salts, e.g. sodium or potassium salts, alkaline earth metal salts, e.g. calcium or magnesium salts; and salts formed with suitable organic ligands, e.g. quaternary ammonium salts.

The present invention includes within its scope pro-drugs of the compounds of Formulas (I)-(II) above. In general, such pr-drugs are functional derivatives of the compounds of Formulas (I)-(II) which are readily convertible in vivo into the required compound of Formulas (I)-(II). Conventional procedures for selecting and preparing suitable pro-drug derivatives are described, for example, in “Design of Prodrugs,” H. Bundgaard, editor, Elsevier, © 1985.

Where the compounds according to the invention have at least one asymmetric center, they may accordingly exist as enantiomers. Where the compounds according the invention possess two or more asymmetric centers, they may additionally exist as diastereoisomers. It is to be understood that all such isomers and mixtures thereof in any proportion are encompassed within the scope of the present invention, including racemic mixtures, single enantiomer or diastereomers, and enantiomerically enriched mixtures.

The invention also provides pharmaceutical compositions comprising one or more compounds of this invention optionally in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation. It is also envisioned that the compounds of the present invention may be incorporated into transdermal patches designed to deliver the appropriate amount of the drug in a continuous fashion.

For preparing solid compositions such as tablets, the principal active ingredient is mixed with a pharmaceutical carrier, e.g., conventional tableting ingredients such as corn starch, lactose, sucrose, sorbitol, talc, stearic acid, magnesium stearate, dicalcium phosphate or gums, mannitol, urea, dextrans, vegetable oils, polyalkylene glycols, ethyl cellulose, poly(vinylpyrrolidone), calcium carbonate, ethyl oleate, isopropyl myristate, benzyl benzoate, sodium carbonate, gelatin, potassium carbonate, silicic acid, and other conventionally employed acceptable carriers. The pharmaceutical dosage form may also contain non-toxic auxiliary substances such as emulsifying, preserving, or wetting agents, and the like. Conventionally the dry formulation is admixed with a pharmaceutical diluent, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be easily subdivided into equally effective unit dosage forms such as tablets, pills and capsules. This solid preformulation composition is then subdivided into unit dosage forms of the type described above containing from about 0.1 to about 500 mg of the active ingredient of the present invention. Typical unit dosage forms contain from about 1 to about 100 mg, for example, 1, 2, 5, 10, 25, 50 or 100 mg, of the active ingredient.

The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which, serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Solid dosage forms may also contain any number of additional non-active ingredients known to the art, including excipients, lubricants, dessicants, binders, colorants, disintegrating agents, dry flow modifiers, preservatives, and the like.

The liquid forms in which the novel compositions of the present invention may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium caboxymethylcellulose, methylcellulose, polyvinylpyrrolidone or gelatin.

In the treatment of cancer in humans, suitable dosage level is from about 0.01 to about 250 mg/kg per day, preferably about 0.05 to about 100 mg/kg per day, and especially about 0.05 to about 5 mg/kg per day. The compounds may be administered on a regimen of 1 to 4 times per day, or on a continuous basis via, for example, the use of a transdermal patch, or 1-4 times every 28 days intravenously, similar to other cancer therapy treatment regimens.

The above-described compounds being effective to inhibit the growth of cancer cells, the compounds are suitable for the therapeutic treatment of neoplastic conditions in mammals, including humans. Cancer cell growth inhibition at pharmacologically-acceptable concentrations has been shown in human breast cancer, brain cancer, lung cancer, colon cancer, and prostate cancer cell lines.

Administration of the subject compounds and compositions to a human or non-human patient can be accomplished by any means known. The preferred administration route is parenteral, including intravenous administration, intraarterial administration, intratumor administration, intramuscular administration, intraperitoneal administration, and subcutaneous administration in combination with a pharmaceutical carrier suitable for the chosen administration route. The treatment method is also amenable to oral administration.

It must be noted, as with all pharmaceuticals, the concentration or amount of the polyamine administered will vary depending upon the severity of the ailment being treated, the mode of administration, the condition and age of the subject being treated, and the particular compound or combination of compounds being used. Thus, the dosages noted previously are guidelines only. Dosages above and below the stated ranges are explicitly encompassed by the invention. The dose administered is ultimately at the discretion of the medical or veterinary practitioner.

Liquid forms for ingestion can be formulated using known liquid carriers, including aqueous and non-aqueous carriers, suspensions, oil-in-water and/or water-in-oil emulsions, and the like. Liquid formulation may also contain any number of additional non-active ingredients, including colorants, fragrance, flavorings, viscosity modifiers, preservatives, stabilizers, and the like.

For parenteral administration, the subject compounds may be administered as injectable dosages of a solution or suspension of the compound in a physiologically-acceptable diluent or sterile liquid carrier such as water or oil, with or without additional surfactants or adjuvants. An illustrative list of carrier oils would include animal and vegetable oils (peanut oil, soy bean oil), petroleum-derived oils (mineral oil), and synthetic oils. In general, for injectable unit doses, water, saline, aqueous dextrose and related sugar solutions, and ethanol and glycol solutions such as propylene glycol or polyethylene glycol are preferred liquid carriers.

Other objects, features and advantages of the present invention will become apparent after review of the specification, claims and drawings.

### BRIEF DESCRIPTION OF FIGURES

FIG. 1 depicts the decomposition and metabolism of glucosinolates, as exemplified by glucoraphanin 1. Deglycosylation of glucosinolates 1 by myrosinase and subsequent rearrangement yields isothiocyanates, as exemplified by sulforaphane 2 that are further metabolized through the mercapturic acid pathway to yield cysteine-conjugates 3 which are moderate HDAC inhibitors.

FIG. 2 is a schematic diagram depicting the general tripartate structure found in known, biologically-relevant histone deacetylase (HDAC) inhibitors. The majority of HDAC inhibitors are characterized as having three key elements: an enzyme-binding pharmacophore, a recognition affinity cap, and an intervening linker of specified length and limited functionality. Specifically shown in FIG. 2 are trapoxin B (4), trichostatin A (5), suberoylanilide hydroxamic acid (SAHA) (6), and pyroxamide (7).

FIGS. 3A and 3B are histograms presenting the IC₅₀ data from Calcein AM (FIG. 3A) and CellTiter Glo-brand (FIG. 3B) high-throughput cytotoxicity assays. Reciprocal IC₅₀ values are displayed for clarity, with the current figure representing an IC₅₀ range of 1.18 μM (compound 22, NCI/ADR RES cells) to >50 μM (e.g., compound 15, all cell lines). Compounds exhibiting IC₅₀ values greater than 50 μM were considered to be non-inhibitory (1/IC₅₀=0) in all cell lines, with the exception of the NmuMG where 200 μM was used. The IC₅₀ value for each library member represents at least three replicates of dose-response experiments conducted over five concentrations at 2-fold dilutions. IC₅₀ values and corresponding error values can be found in Table 1. The five library member “hits” are shown at the top of FIG. 3A for structural comparison. FIG. 3A: Reciprocal IC₅₀ values calculated using the Calcein AM assay. Live cells were distinguished by the presence of a ubiquitous intracellular enzymatic activity that converts the non-fluorescent, cell-permeable molecule calcein AM to the intensely fluorescent molecule calcein, which is retained within live cells. FIG. 3B: Reciprocal IC₅₀ values calculated using the CellTiter-Glo-brand assay (Promega, Madison, Wis.). Live cells were observed by fluorescence via the enzymatic action of luciferase on luciferin, a process which is dependent and proportional to the cellular concentration of ATP. Du145=human prostate carcinoma; HCT-116=human colon carcinoma; Hep3B=human liver carcinoma; SF-268=human CNS glioblastoma; SK-OV-3=human ovary adenocarcinoma; NCI/ADR RES=human breast carcinoma; NCI-H460=human breast carcinoma; MCF7=human breast carcinoma; NmuMG=mouse mammary normal epithelial cells.

FIG. 4 is a histogram presenting IC₅₀ data from the MTT cytotoxicity assay in HT-29 cells. Reciprocal IC₅₀ values are displayed for clarity and range from 17.02 μM (28) to >50 μM (e.g., 15). Compounds exhibiting IC₅₀ values greater than 50 μM were considered to be non-inhibitory (1/IC₅₀=0). The IC₅₀ value for each library member represents at least twelve replicates of dose-response experiments conducted over five concentrations. IC₅₀ values and corresponding error values can be found in Table 1. In this assay, live cells were distinguished by the intracellular enzymatic activity that converts the cell-permeable molecule MTT to strongly colored formazan crystals, which are retained within live cells and absorb light at 570 nm HT-29=human liver carcinoma. * IC₅₀>40 μM.

FIG. 5 is a histogram showing IC₅₀ data from the Calcein AM cytotoxicity assay for isothiocyanate compounds 2 and 12-25 as described herein against various neoplastic cell lines. Error bars represent standard error. The neoplastic cell types in the screen are the same as those used in FIGS. 3A and 3B.

FIG. 6 is a histogram showing IC₅₀ data from the Calcein AM cytotoxicity assay isothiocyanate compounds 26-40 as described herein against various neoplastic cell lines. Error bars represent standard error. The neoplastic cell types in the screen are the same as those used in FIGS. 3A and 3B.

FIG. 7 is a histogram showing IC₅₀ data from the CellTiter-Glo assay for isothiocyanate compounds 2 and 12-25 as described herein against various neoplastic cell lines. Error bars represent standard error. The neoplastic cell types in the screen are the same as those used in FIGS. 3A and 3B.

FIG. 8 is a histogram showing IC₅₀ data from the CellTiter-Glo assay isothiocyanate compounds 26-40 as described herein against various neoplastic cell lines. Error bars represent standard error. The neoplastic cell types in the screen are the same as those used in FIGS. 3A and 3B.

### DETAILED DESCRIPTION OF THE INVENTION

### Abbreviations and Definitions

Unless defined otherwise, all technical and scientific terms used herein have the same meanings as commonly understood by one of ordinary skill in the art of pharmaceutical chemistry, pharmacology, biochemistry, and enzymology.

As used herein and in the claims, the singular forms “a”, “an”, and “the” include plural reference unless the context clearly dictates otherwise. Thus, for example, reference to “a HDAC inhibitor” includes a plurality of such inhibitors and equivalents thereof known to those skilled in the art, and so forth. The terms “a” (or “an”), “one or more” and “at least one” are used interchangeably herein. As used herein, the terms “comprising,” “including,” “characterized by,” and “having,” are synonymous and indicated an “open-ended” construction.

Although any methods and materials similar or equivalent to those described herein can be used in the practice or testing of the present invention, the preferred methods and materials are now described. All cited publications are incorporated herein by reference for the purpose of describing and disclosing the chemicals, cell lines, vectors, animals, instruments, statistical analysis and methodologies which are reported in the publications which might be used in connection with the invention. Nothing herein is to be construed as an admission that the invention is not entitled to antedate such disclosure by virtue of prior invention.

The practice of the present invention will employ, unless otherwise indicated, conventional techniques of organic chemistry, molecular biology, microbiology, recombinant DNA, and immunology, which are within the skill of the art. Such techniques are explained fully in the literature. See, for example, Molecular Cloning: A Laboratory Manual, 2nd Ed., ed. by Sambrook, Fritsch and Maniatis (Cold Spring Harbor Laboratory Press, 1989); DNA Cloning, Volumes I and II (D. N. Glover ed., 1985); Oligonucleotide Synthesis (M. J. Gait ed., 1984); Mullis et al., U.S. Pat. No. 4,683,195; Nucleic Acid Hybridization (B. D. Hames & S. J. Higgins eds. 1984); Transcription and Translation (B. D. Hames & S. J. Higgins eds., 1984); Culture Of Animal Cells (R. I. Freshney, Alan R. Liss, Inc., 1987); Immobilized Cells And Enzymes (IRL Press, 1986); B. Perbal, A Practical Guide to Molecular Cloning (1984); the treatise, Methods In Enzymology (Academic Press, Inc., N.Y.); Gene Transfer Vectors For Mammalian Cells (J. H. Miller and M. P. Calos, eds., 1987, Cold Spring Harbor Laboratory); Methods In Enzymology, Vols. 154 and 155 (Wu et al., eds), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986).

In order to provide a clearer and consistent understanding of the specification and claims, including the scope to be given such terms, the following definitions are provided.

A “therapeutically effective amount” of an active agent is the amount effective to inhibit the growth of neoplastic cells (i.e., tumors, both benign and malignant) in vivo when the compound is administered via any given route of administration. Thus, the therapeutically effective amount may vary considerably based upon the method of administration (oral, intravenous, inhalation, etc.) An effective amount of a compound of Formula I or II, or an analog thereof, is thus the amount of one or more of these substances, with or without a pharmaceutically suitable carrier, that is effective to inhibit the growth of neoplastic cells when administered to a patient suffering from (or suspected of suffering from) such neoplastic growth.

Abbreviations used herein include:

BITC=benzyl isothiocyanate

m-CPBA=meta-chloroperoxybenzoic acid.

Di-2PTC=di-(2-pyridyl)-thionocarbonate

DIEA=diisopropylethylamine

DMF=dimethylformamide

DMSO=dimethylsulfoxide

EDTA=ethylenediamine tetraacetic acid

EtOAc=ethyl acetate

Et₂O=diethyl ether

EtOH=ethanol

HDAC=histone deacetylase

HIF-1=hypoxia-inducible factor 1

HRMS (EI-EMM)=high-resolution mass spectrum (electron impact-exact mass measurement)

HRMS (ESI-EMM)=high-resolution mass spectrum (electrospray ionization-exact mass measurement)

ITC=isothiocynate

LRMS=low-resolution mass spectrum

MTT=2-(4,5-dimethyl-2-thiazolyl)-3,5-diphenyl-2H-tetrazolium bromide

NMR=nuclear magnetic resonance

PEITC=phenethyl isothiocyanate

6-PEITC=6-phenylhexyl isothiocyanate and s

SAHA=suberoylanilide hydroxamic acid

THF=tetrahydrofuran

VEGF=vascular endothelial growth factor (VEGF)

VHL=von Hippel Lindau tumor suppressor protein

### Histone Deacetylases:

Angiogenesis, hypoxia and hypoxia-inducible factor 1 (HIF-1) are coupled through the actions of histone deacetylases 1 (HDAC1). The growth of new blood vessels into a cancer (angiogenesis) is required for continued growth of the tumor mass beyond 1-2 mm3. Increased numbers of blood vessels in breast cancer, and other cancers as well, correlates closely with metastasis and poor prognosis. Tumor hypoxia is a major inducer of vascular endothelial growth factor (VEGF) gene expression (Kim et al., 2001, Nature Medicine 7: 437-443). VEGF expression is under the control of HIF-1, a heterodimeric transcription factor recognized as the key regulator of the hypoxia response in a variety of cell types (Kim et al. (2001) Nat. Med. 7: 437-443; Semenza (2000) Cancer and Metastatis Rev. 19: 56-65; Semenza, (2001) Curr. Op. Cell Biol. 13: 167-171; Ratcliffe et al. (2000) Nat. Med. 6: 1315-1316). Composed of HIF-1α and HIF-1β, HIF-1 activates the transcription of genes encoding angiogenic growth factors and vasomotor regulators. HIF-1 also regulates the expression of molecules involved in matrix modeling, iron transport/regulation and apoptosis/cell) proliferation. HIF-1α is constitutively expressed, whereas HIF-1β is induced by exposure of cells to hypoxia or growth factors. Importantly, HIF expression levels are characteristically increased in many cancerous tumor types as are a number of reductases (Saramaki et al. (2001) Cancer Gen. and Cytogen. 128: 31-34; Huss et al. (2001) Cancer Res. 61: 2736-2743; Cvetkovic et al. (2001) Urology 57: 821-825).

Under normoxic conditions, HIF-1α is degraded by the ubiquitin-proteosome system. This process relies upon the von Hippel Lindau (VHL) tumor suppressor protein; interaction with HIF-1α affords the recognition component of an E3 ubiquitin ligase complex (Kim et al. (2001) Nat. Med. 7: 437-443). Hypoxia-associated reduction of VHL levels leads to HIF-1α accumulation and subsequent overexpression of proangiogenic (metastasis-associated) agents. Hypoxia and HIF-1α overexpression are hallmarks of many tumor types, particularly prostate carcinomas (Saramaki et al. (2001) Cancer Gen. and Cytogen. 128: 31-34; Cvetkovic et al. (2001) Urology 57: 821-825).

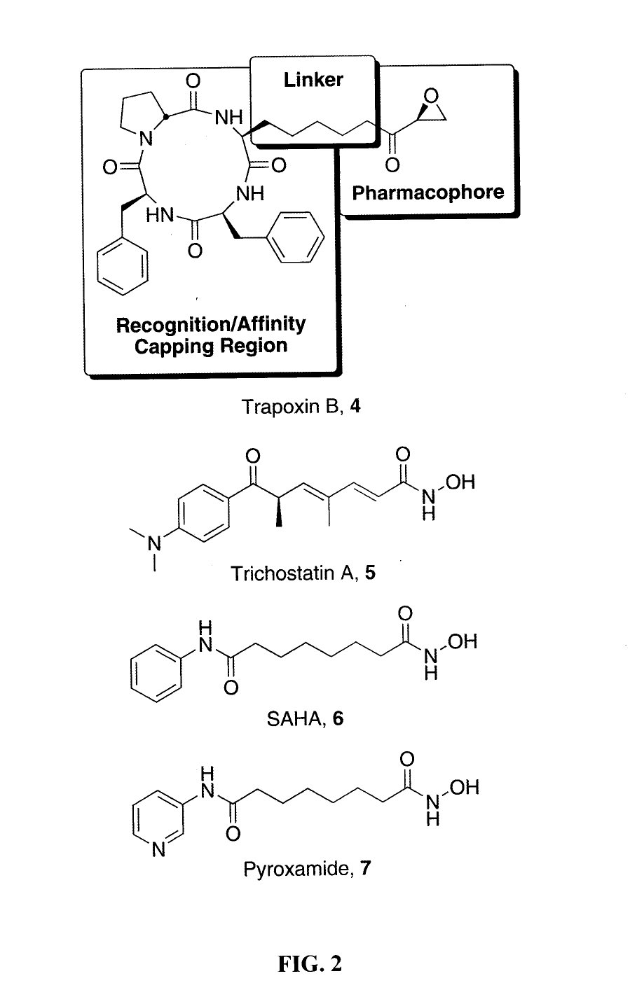

A tremendous amount of structure-activity relationship (SAR) data is now available for a wide array of HDAC inhibitors (19-20). The majority of effective HDAC inhibitors are characterized by a tripartate structure (21-24) that, to a certain extent, mimics the native substrate of HDAC action, ε-N-acetyl Lys (23). The enzyme affinity “cap” is connected to an enzyme active site binding/inactivating group via a linker devoid of elaborate functionality (see FIG. 2) (19-20). The established importance of linker length and linearity and the scarcity of high resolution structural information have led to the examination of broadly different cap structures (21-24). The resounding conclusion of this work is that ideal cap groups are typically very lipophilic, often containing one or more phenyl rings (19-24).

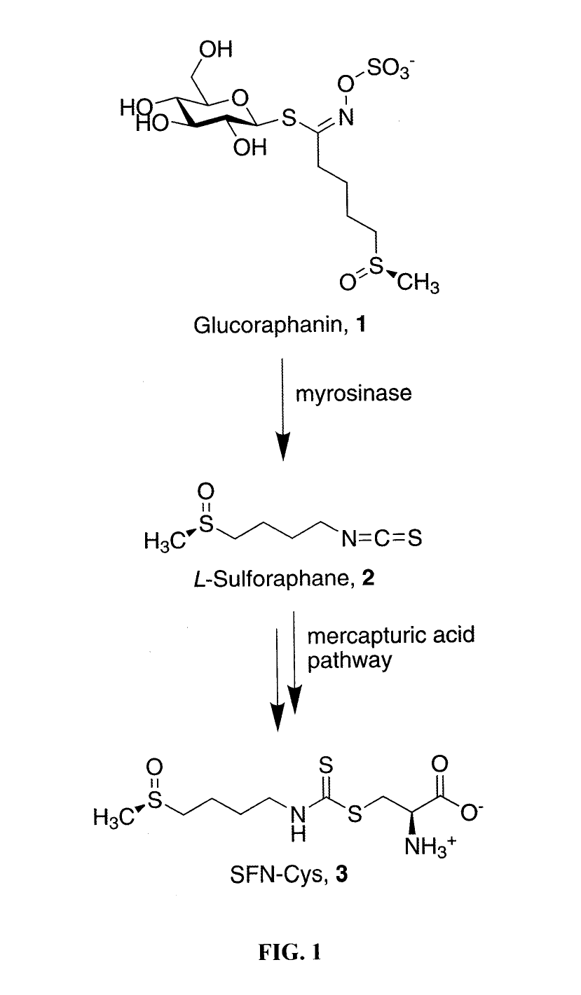

Notably, compound 3 (see FIG. 1) does not contain many of the structural features common among many potent MAC inhibitors. Following the structural hypothesis established by Dashwood, the 4-(methylsulfinyl)-butyl moiety of 3 is vastly more polar than the cap groups of established MAC inhibitors 4-7 depicted in FIG. 2. Moreover, reports from the Yu laboratory have shown that significantly different degrees of lung tumor prevention are observed with phenethyl (PEITC) and 6-phenylhexyl (6-PEITC) and benzyl (BITC) isothiocyanates (17, 24). The present inventors thus suspected that 3 contains a sub-optimal linker length connecting the affinity cap group and the pharmacophore.

It was hypothesized that the combination of the non-optimal features of 3 as a HDAC inhibitor may be responsible for its relatively low levels of activity. It was further hypothesized that increased potency as a HDAC inhibitor would correlate to enhanced chemopreventive properties of the parent isothiocyanate. To test this hypothesis, the inventors constructed a panel of isothiocyanates whose functionality more-closely resembles known HDAC inhibitors. Resulting from these efforts, the inventors have identified multiple ITCs with improved potency and selectivity for cancerous cells relative to L-sulforaphane 2. And, while not being bound to any particular underlying biological mechanism, several trends in the structure-activity relationships of the ITCs have been observed that suggest that the chemopreventive properties of ITCs arises, in part, from their HDAC-inhibitor activity.

Thus, increased potency as a HDAC inhibitor correlates with enhanced chemopreventive properties of the parent ITC. Using a small library of synthetic isothiocyanates, several novel ITCs with bioactivities equal to or superior to sulforaphane have been identified. Also, the effects that the oxidation state of the sulfur, linker length, lipophilicity, and stereochemistry have on cytotoxicity of ITCs have been identified. This information both expands upon the structure/activity database for ITCs and is supportive of trends observed among HDAC inhibitors, further implicating the capability of ITCs to act as precursors of HDAC inhibitors.

### Experimental Procedures

Chemicals and Reagents. All chemicals and reagents were purchased from Sigma-Aldrich (Milwaukee, Wis.) and used as received, unless specially noted. Anhydrous CH₂Cl₂, DMF, and THF are Optima-grade solvents purchased from Sigma-Aldrich (Milwaukee, Wis.) dispensed using a Glass Contour Solvent Dispensing System. Instrumentation. NMR spectra were acquired using Varian Unity Inova 400 and 500 MHz spectrometers with solvent as the internal reference. ESI mass spectra were acquired using an Agilent 1100 HPLC-MSD SL quadrupole mass spectrometer. High-resolution mass spectra of synthetic intermediates of sulforaphane and isothiocyanates were acquired at the University of Wisconsin Department of Chemistry Analytical Instrumentation Facility using electrospray ionization.

Syntheses of D,L-Sulforaphane and Erysolin. The syntheses of D,L-sulforaphane and erysolin were modified from a previously-reported procedure according to Scheme 1 (25).

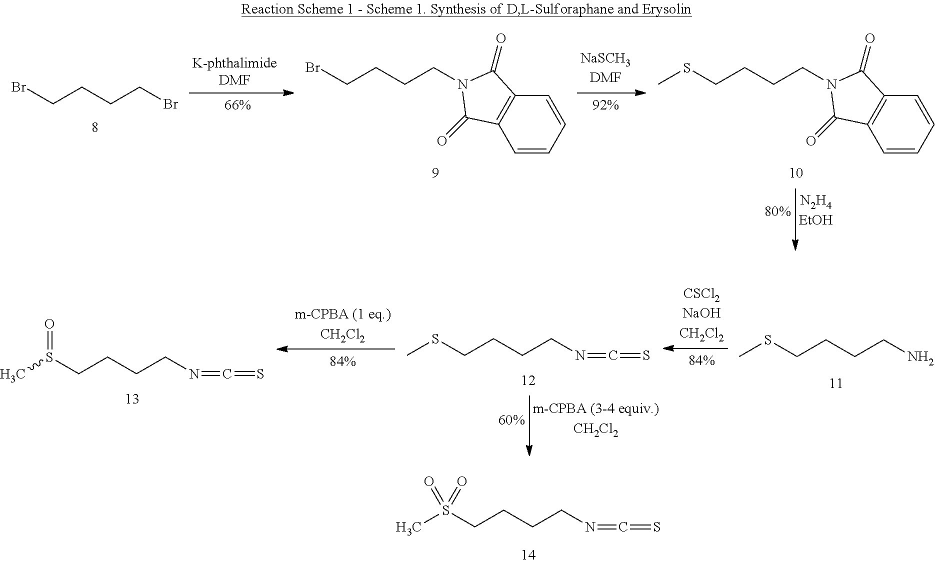

Procedures and spectral characterization of intermediates are described in the following paragraphs. Starting from 1,4,-dibromobutane, D,L-sulforaphane was obtained in 34% overall yield after five steps.

Syntheses of Isothiocyanates. Isothiocyanates were synthesized from their corresponding commercially-available primary amines according to one of two general procedures according to Scheme 2 (25, 26).

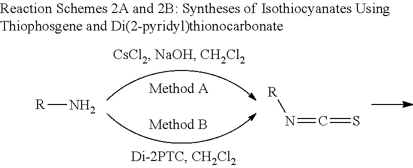

General Method A: Isothiocyanate Installation Using Thiophosgene. The following procedure was adapted from that previously reported by Vermeulen, et al. (25). A 0.50 M solution of thiophosgene (3 equiv) in anhydrous CH₂Cl₂ was chilled to 0° C. under argon. A solution of the primary amine in anhydrous CH₂Cl₂ (1 mL/mmol) was added. If the hydrochloride salt of the amine was used, it was first neutralized using diisopropylethylamine (DIEA, 1-2 equiv). Finely-crushed NaOH (3 equiv) was then added and the resulting solution was allowed to warm to ambient temperature over 3 h. Products were concentrated in vacuo and any resulting solids were removed by filtration.

General Method B: Isothiocyanate Installation Using Di-(2-pyridyl)-thionocarbonate (Di-2PTC). The following procedure was adapted from that previously reported by Park, et al. (26). The primary amine was dissolved in anhydrous CH₂Cl₂ (14.5 mL/mmol) at ambient temperature and Di-2PTC (1 equiv) was added. The reaction was stirred under argon for 24 hours, followed by solvent removal in vacuo.

### Syntheses of D,L-Sulforaphane and Erysolin:

Compound 9: 2-(4-bromobutyl)isoindoline-1,3-dione. The following procedure was adapted from that previously reported by Vermeulen, et al. (25). 1,4-Dibromobutane (4.400 mL, 36.457 mmols) was dissolved in anhydrous DMF (52 mL) and the resulting solution was chilled to 0° C. under argon. After 15 min, potassium phthalimide (3.459 g, 18.672 mmols) was slowly added to the stirring solution and the reaction was allowed to warm to ambient temperature under argon over 18 h. The reaction was concentrated in vacuo and co-stripped with anhydrous THF several times. Products were dissolved in 1:1 H₂O:EtOAc (200 mL) and the aqueous phase was extracted with EtOAc (3×100 mL). Combined organics were washed with brine, dried over Na₂SO₄, and filtered through a celite plug prior to concentration in vacuo. Silica gel chromatography (3:1 Hexane:EtOAc) and subsequent concentration afforded 3.466 g 9 as a white solid (66% yield). ¹H NMR (CDCl₃) δ 7.85 (dd, J=5.4, 3.1 Hz, 2H), 7.73 (dd, J=5.4, 3.0 Hz, 2H), 3.73 (t, J=6.7 Hz, 2H), 3.45 (t, J=6.4 Hz, 2H), 1.89 (m, 4H). ¹³C NMR (CDCl₃) δ 168.5, 134.1, 132.2, 123.4, 37.1, 32.9, 30.0, 27.4. HRMS (ESI-EMM) calc'd for [M+Na]+m/z 303.9949. found 303.9936.

Compound 10: 2-(4-(methylthio)butyl)isoindoline-1,3-dione. The following procedure was adapted from that previously reported by Vermeulen, et al. (25). Sodium thiomethoxide (3.808 g, 54.328 mmols) was dissolved in anhydrous DMF (40 mL) and chilled to 0° C. under argon. To this was added a solution of 9 (13.700 g, 48.559 mmols) in anhydrous DMF (95 mL). After 15 minutes at 0° C., the reaction was allowed to warm to ambient temperature over 18 h. The resulting solution was slowly poured into a stirring, ice-chilled bath of deionized water (800 mL). The precipitate was collected by filtration, washed with cold water, and redissolved CH₂Cl₂ (400 mL). Organics were washed with brine, dried over Na₂SO₄, and concentrated in vacuo to afford 11.1 g 10 as pinkish-white crystals (92% yield). ¹H NMR (CDCl₃) δ 7.84 (dd, J=5.4, 3.1 Hz, 2H), 7.72 (dd, J=5.4, 3.0 Hz, 2H), 3.71 (t, J=7.1 Hz, 2H), 2.54 (t, J=7.3 Hz, 2H), 2.09 (s, 3H), 1.80 (m, 2H), 1.65 (m, 2H). ¹³C NMR (CDCl₃) δ 168.5, 134.0, 132.2, 123.3, 37.6, 33.7, 27.8, 26.5, 15.6. HRMS (ESI-EMM) calc'd for [M+Na]+m/z 272.0721. found 272.0727.

Compound 11: 4-(methylthio)butan-1-amine. The following procedure was adapted from that previously reported by Vermeulen, et al. (25). Compound 10 (2.003 g, 8.035 mmols) was dissolved in absolute EtOH (48 mL) and hydrazine monohydrate (520 μL, 537 mg, 10.727 mmols) was added. This solution was heated to reflux for 3 hours, then cooled to 0° C. to fully-precipitate the solid. The solid was removed by filtration and was washed excessively with anhydrous Et₂O (1 L). The filtrates were combined and concentrated in vacuo. Distillation at reduced pressure (6 mm Hg), b.p. 55° C.) afforded 762 mg 11 as a colorless oil (80% yield). ¹H NMR (CDCl₃) δ 2.72 (t, J=6.7 Hz, 2H), 2.52 (t, J=7.4 Hz, 2H), 2.10 (s, 3H), 1.64 (m, 2H), 1.55 (m, 2H), 1.33 (bs, 2H). ¹³C NMR (CDCl₃) δ 42.1, 34.4, 33.2, 26.7, 15.7. LRMS (ESI) calc'd for [M+H]+m/z 120.1. found 120.1.

Compound 12: 1-isothiocyanato-4-(methylthio)butane (trivial name: erucin). The following procedure was adapted from that previously reported by Vermeulen, et al. (25). Thiophosgene (1.380 mL, 18.099 mmols) was dissolved in anhydrous CH₂Cl₂ (41 mL) and chilled to 0° C. under argon. Compound 11 (698 mg, 5.856 mmols) and NaOH (607 mg, 15.166 mmols) were added in sequence and the solution was allowed to warm to ambient temperature over 3.5 h. The resulting solution was concentrated in vacuo and filtered to remove any solid. Silica gel chromatography (3:1 Hexane:CH₂Cl₂) and subsequent concentration afforded 795 mg 12 as a orange oil (84% yield). ¹H NMR (CDCl₃) δ 3.55 (t, J=6.4 Hz, 2H), 2.53 (t, J=6.9 Hz, 2H), 2.09 (s, 3H), 1.87-1.68 (m, 4H). ¹³C NMR (CDCl₃) δ 129.4, 44.4, 32.8, 28.5, 25.4, 14.9. HRMS (EI-EMM) calc'd for [M]+m/z 161.0333. found 161.0337.

Compound 13: 1-isothiocyanato-4-(methylsulfinyl)butane (trivial name: D,L-sulforaphane). The following procedure was adapted from that previously reported by Vermeulen, et al. (25). Compound 12 (795 mg, 4.930 mmols) was dissolved in anhydrous CH₂Cl₂ (7.0 mL) under argon. To this was slowly added a solution of m-CPBA (934 mg, 5.410 mmols) in anhydrous CH₂Cl₂ (6.25 mL). After 2 h, the reaction was diluted with CH₂Cl₂ and the organics were washed with sat'd. NaHCO₃, brine, and dried over Na₂SO₄ prior to concentration in vacuo. Silica gel chromatography (2:1 CH₂Cl₂:CH₃CN) and subsequent concentration afforded 735 mg 13 as a light yellow oil (84% yield). ¹H NMR (CDCl₃) δ 3.58 (t, J=6.2 Hz, 2H), 2.71 (m, 2H), 2.58 (s, 3H), 1.95-1.81 (m, 4H). ¹³C NMR (CDCl₃) δ 129.8, 52.9, 44.3, 38.3, 28.5, 19.6. HRMS (ESI-EMM) calc'd for [M+Na]+m/z 200.0180. found 200.0172.

Compound 14: 1-isothiocyanato-4-(methylsulfonyl)butane (trivial name: erysolin). The following procedure was adapted from that previously reported by Vermeulen, et al. (25). Compound 12 (283 mg, 1.754 mmols) was dissolved in anhydrous CH₂Cl₂ (2.5 mL) under argon. To this was slowly added a solution of m-CPBA (964 mg, 5.586 mmols) in anhydrous CH₂Cl₂ (5.0 mL). After 2 h, the reaction was diluted with CH₂Cl₂ and the organics were washed with sat'd. NaHCO₃, brine, and dried over Na₂SO₄ prior to concentration in vacuo. Silica gel chromatography (CH₂Cl₂) and subsequent concentration afforded 203 mg 14 as an off-white solid (60% yield). ¹H NMR (CDCl₃) δ 3.56 (t, J=6.1 Hz, 2H), 3.01 (t, J=7.8 Hz, 2H), 2.86 (s, 3H), 1.90 (m, 2H), 1.81 (m, 2H). ¹³C NMR (CDCl₃) δ 130.4, 53.5, 44.4, 40.6, 28.4, 19.6. HRMS (EI-EMM) calc'd for [M]+m/z 193.0231. found 193.0230.

### Syntheses of Isothiocyanates

General Method A: Isothiocyanate Installation Using Thiophosgene. The following procedure was adapted from that previously reported by Vermeulen, et al. (25). A 0.50 M solution of thiophosgene (3 equiv) in anhydrous CH₂Cl₂ was chilled to 0° C. under argon. A solution of the primary amine in anhydrous CH₂Cl₂ (1 mL/mmol) was added. If the hydrochloride salt of the amine was used, it was first neutralized using diisopropylethylamine (DIEA, 1-2 equiv). Finely-crushed NaOH (3 equiv) was then added and the resulting solution was allowed to warm to ambient temperature over 3 h. Products were concentrated in vacuo and any resulting solids were removed by filtration.

General Method B: Isothiocyanate Installation Using Di(2-pyridyl)-thionocarbonate. The following procedure was adapted from that previously reported by Park, et al. (26). The primary amine was dissolved in anhydrous CH₂Cl₂ (14.5 mL/mmol) at ambient temperature and di(2-pyridyl)thionocarbonate (1 equiv) was added. The reaction was stirred under argon for 24 hours, followed by solvent removal in vacuo.

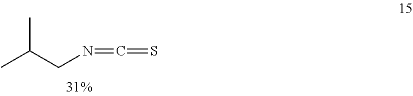

Compound 15: 1-isothiocyanato-2-methylpropane. Compound 15 was synthesized by Method A from thiophosgene (206 μL, 311 mg, 2.705 mmols), isobutylamine (96 μL, 70 mg, 0.957 mmols), and NaOH (133 mg, 3.324 mmols). Silica gel chromatography (5:1 Hexane:CH₂Cl₂) and subsequent concentration afforded 31 mg 15 as an orange oil (28% yield). ¹H NMR (CDCl₃) δ 3.34 (d, J=6.2 Hz, 2H), 2.00 (nonet, J=6.7 Hz, 1H), 1.01 (d, J=6.7 Hz, 6H). ¹³C NMR (CDCl₃) δ 129.8, 52.6, 29.8, 19.9.

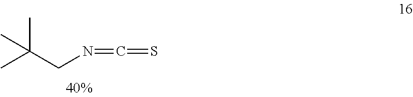

Compound 16: 1-isothiocyanato-2,2-dimethylpropane. Compound 16 was synthesized by Method A from thiophosgene (196 μL, 296 mg, 2.574 mmols), neopentylamine (112 μL, 83 mg, 0.854 mmols), and NaOH (137 mg, 3.424 mmols). Silica gel chromatography (5:1 Hexane:CH₂Cl₂) and subsequent concentration afforded 55 mg 16 as a light-orange oil (40% yield). ¹H NMR (CDCl₃) δ 3.26 (s, 2H), 1.02 (s, 9H). ¹³C NMR (CDCl₃) δ 57.3, 33.5, 27.1.

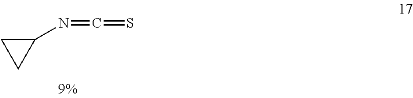

Compound 17: isothiocyanatocyclopropane. Compound 17 was synthesized by Method A from thiophosgene (903 μL, 1.362 g, 11.845 mmols), cyclopropylamine (271 μL, 221 mg, 3.871 mmols), and NaOH (488 mg, 12.197 mmols). Silica gel chromatography (5:1 Hexane:CH₂Cl₂) and subsequent concentration afforded 35 mg 17 as an orange oil (9% yield). ¹H NMR (CDCl₃) δ 2.89 (tt, J=7.0, 3.8 Hz, 1H), 0.93-0.87 (m, 2H), 0.87-0.81 (m, 2H). ¹³C NMR (CDCl₃) δ 126.7, 25.5, 8.3.

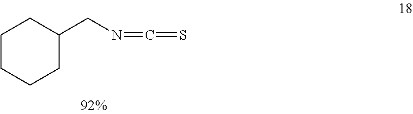

Compound 18: (isothiocyanatomethyl)cyclohexane. Compound 18 was synthesized by Method A from thiophosgene (206 μL, 311 mg, 2.705 mmols), cyclohexylmethylamine (125 μL, 109 mg, 0.963 mmols), and NaOH (131 mg, 3.274 mmols). Silica gel chromatography (3:1 Hexane:CH₂Cl₂) and subsequent concentration afforded 138 mg 18 as an orange oil (92% yield). ¹H NMR (CDCl₃) δ 3.33 (d, J=6.3 Hz, 2H), 1.80-1.71 (m, 4H), 1.17-1.59 (m, 2H), 1.32-1.07 (m, 3H), 1.06-0.94 (m, 2H). ¹³C NMR (CDCl₃) δ 129.5, 51.3, 38.7, 30.4, 26.1, 25.7. HRMS (EI-EMM) calc'd for [M]+m/z 155.0769. found 155.0771.

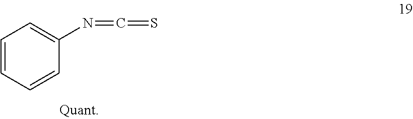

Compound 19: 1-isothiocyanatobenzene (PITC). Compound 19 was synthesized by Method A from thiophosgene (182 μL, 274 mg, 2.383 mmols), aniline (87 μL, 89 mg, 0.956 mmols), and NaOH (127 mg, 3.174 mmols). Silica gel chromatography (25:1 Hexane: CH₂Cl₂) and subsequent concentration afforded 19 as a colorless oil in quantitative yield. ¹H NMR (CDCl₃) δ 7.33 (m, 2H), 7.26 (m, 1H), 7.20 (m, 2H). ¹³C NMR (CDCl₃) δ 135.5, 131.4, 129.7, 127.5, 125.9. HRMS (EI-EMM) calc'd for [M]+m/z 135.0143. found 135.0149.

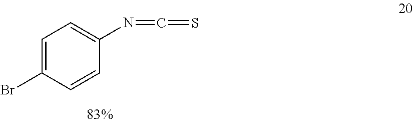

Compound 20:1-bromo-4-isothiocyanatobenzene. Compound 20 was synthesized by Method A from thiophosgene (208 μL, 314 mg, 2.727 mmols), 4-bromoaniline (163 mg, 0.949 mmols), and NaOH (144 mg, 3.599 mmols). Silica gel chromatography (25:1 Hexane:CH₂Cl₂) and subsequent concentration afforded 168 mg 20 as a white solid (83% yield). ¹H NMR (CDCl₃) δ 7.46 (d, J=8.7 Hz, 2H), 7.08 (d, J=8.7 Hz, 2H). ¹³C NMR (CDCl₃) δ 137.1, 132.9, 130.7, 127.3, 120.9. HRMS (EI-EMM) calc'd for [M]+m/z 212.9248. found 212.9245.

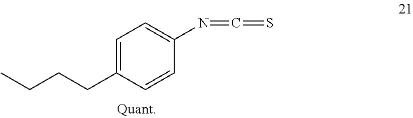

Compound 21: 1-butyl-4-isothiocyanatobenzene. Compound 21 was synthesized by Method A from thiophosgene (208 μL, 314 mg, 2.727 mmols), 4-butylaniline (150 μL, 140 mg, 0.938 mmols), and NaOH (123 mg, 3.074 mmols). Silica gel chromatography (25:1 Hexane:CH₂Cl₂) and subsequent concentration afforded 21 as a light-yellow oil in quantitative yield. ¹H NMR (CDCl₃) δ 7.16 (d, J=8.6 Hz, 2H), 7.14 (d, J=8.6 Hz, 2H), 2.62 (t, J=7.8 Hz, 2H), 1.60 (m, 2H), 1.37 (sextet, J=7.4 Hz, 2H), 0.95 (t, J=7.4 Hz, 3H). ¹³C NMR (CDCl₃) δ 142.7, 134.8, 129.6, 128.7, 125.7, 35.4, 33.5, 22.4, 14.0. HRMS (EI-EMM) calc'd for [M]+m/z 191.0769. found 191.0777.

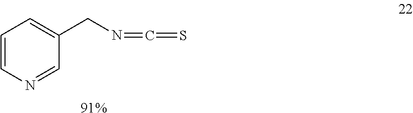

Compound 22: 3-(isothiocyanatomethyl)pyridine. Compound 22 was synthesized by Method B from 3-picolylamine (140 μL, 150 mg, 1.387 mmols), and di(2-pyridyl)thionocarbonate (325 mg, 1.399 mmols). Silica gel chromatography (1:2 Hexane:EtOAc) and subsequent concentration afforded 190 mg 22 as a yellow oil (91% yield). ¹H NMR (CDCl₃) δ 8.62 (d, J=4.4 Hz, 1H), 8.59 (s, 1H), 7.70 (dt, J=7.9, 1.718 Hz, 1H), 7.36 (dd, J=7.8, 4.8 Hz, 1H), 4.76 (s, 2H). ¹³C NMR (CDCl₃) δ 149.7, 148.2, 134.6, 133.7, 130.1, 123.7, 46.4. HRMS (EI-EMM) calc'd for [M]+m/z 150.0252. found 150.0248.

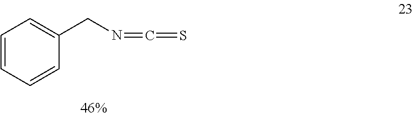

Compound 23: 1-(isothiocyanatomethyl)benzene (BITC). Compound 23 was synthesized by Method A from thiophosgene (480 μL, 724 mg, 6.295 mmols), benzylamine (200 μL, 196 mg, 1.831 mmols), and NaOH (224 mg, 5.606 mmols). Silica gel chromatography (5:1 Hexane:CH₂Cl₂) and subsequent concentration afforded 127 mg 23 as a orange oil (46% yield). ¹H NMR (CDCl₃) δ 7.34 (m, 5H), 4.68 (s, 2H). ¹³C NMR (CDCl₃) δ 134.1, 131.9, 128.8, 128.2, 126.7, 48.5. HRMS (EI-EMM) calc'd for [M]+m/z 149.0299. found 149.0303.

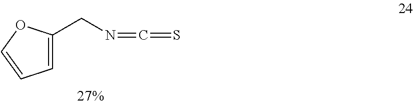

Compound 24: 2-(isothiocyanatomethyl)furan. Compound 24 was synthesized by Method A from thiophosgene (206 μL, 311 mg, 2.705 mmols), furfurylamine (89 μL, 93 mg, 0.958 mmols), and NaOH (125 mg, 3.124 mmols). Silica gel chromatography (3:1 Hexane: CH₂Cl₂) and subsequent concentration afforded 37 mg 24 as an orange oil (27% yield). ¹H NMR (CDCl₃) δ 7.43 (m, 1H), 6.37 (dd, J=3.2, 1.8 Hz, 1H), 6.35 (bd, J=3.2 Hz, 1H), 4.66 (s, 2H). ¹³C NMR (CDCl₃) δ 147.5, 143.4, 135.1, 110.8, 108.9, 42.1. HRMS (EI-EMM) calc'd for [M]+m/z 139.0092. found 139.0092.

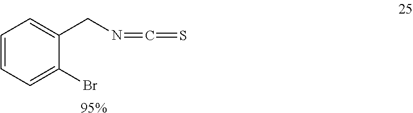

Compound 25: 1-bromo-2-(isothiocyanatomethyl)benzene. Compound 25 was synthesized by Method A from thiophosgene (208 μL, 314 mg, 2.731 mmols), 2-bromo-benzylamine hydrochloride (213 mg, 0.959 mmols), DIEA (250 μL, 186 mg, 1.441 mmols), and NaOH (155 mg, 3.874 mmols). Silica gel chromatography (5:1 Hexane: CH₂Cl₂) and subsequent concentration afforded 209 mg 25 as a reddish-orange oil (95% yield). ¹H NMR (CDCl₃) δ 7.59 (dd, J=8.0, 0.8 Hz, 1H), 7.46 (dd, J=7.7, 1.2 Hz, 1H), 7.38 (td, J=7.4, 0.9 Hz, 1H), 7.23 (td, J=7.7, 1.5 Hz, 1H), 4.81 (s, 2H). ¹³C NMR (CDCl₃) δ 133.8, 133.4, 133.1, 130.1, 128.9, 128.1, 122.5, 49.3. HRMS (EI-EMM) calc'd for [M]+m/z 226.9404. found 226.9405.

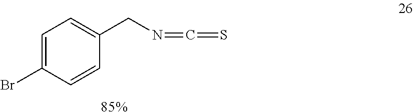

Compound 26: 1-bromo-4-(isothiocyanatomethyl)benzene. Compound 26 was synthesized by Method A from thiophosgene (196 μL, 296 mg, 2.576 mmols), 4-bromo-benzylamine (184 mg, 0.989 mmols), and NaOH (123 mg, 3.074 mmols). Silica gel chromatography (5:1 Hexane:CH₂Cl₂) and subsequent concentration afforded 193 mg 26 as a reddish-orange oil (85% yield). ¹H NMR (CDCl₃) δ 7.51 (d, J=8.4 Hz, 2H), 7.19 (d, J=8.4 Hz, 2H), 4.67 (s, 2H). ¹³C NMR (CDCl₃) δ 133.4, 133.3, 132.2, 128.7, 122.5, 48.3. HRMS (EI-EMM) calc'd for [M]+m/z 226.9404. found 226.9410.

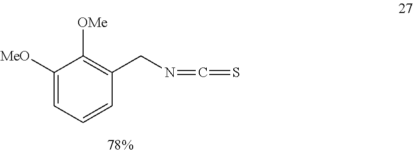

Compound 27: 2-(Isothiocyanatomethyl)-1,2-dimethoxybenzene. Compound 27 was synthesized by Method A from thiophosgene (182 μL, 274 mg, 2.387 mmols), 2,3-dimethoxy-benzylamine (167 mg, 1.001 mmols), and NaOH (130 mg, 3.249 mmols). Silica gel chromatography (4:1 Hexane:CH₂Cl₂ to 3:1 Hexane:CH₂Cl₂) and subsequent concentration afforded 164 mg 27 as a light yellow oil (78% yield). ¹H NMR (CDCl₃) δ 7.08 (t, J=8.0 Hz, 1H), 6.93 (m, 2H), 4.72 (s, 2H), 3.89 (s, 3H), 3.87 (s, 3H). ¹³C NMR (CDCl₃) δ 152.7, 146.6, 131.5, 128.0, 124.4, 120.3, 113.0, 61.0, 55.9, 44.1. HRMS (EI-EMM) calc'd for [M]+m/z 209.0511. found 209.0502.

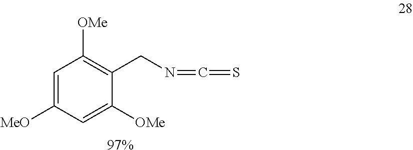

Compound 28: 2-(isothiocyanatomethyl)-1,3,5-trimethoxybenzene. Compound 28 was synthesized by Method A from thiophosgene (182 μL, 274 mg, 2.387 mmols), 2,4,6-trimethoxy-benzylamine (185 mg, 0.938 mmols), and NaOH (123 mg, 3.074 mmols). Silica gel chromatography (1:1 Hexane:CH₂Cl₂ to CH₂Cl₂) and subsequent concentration afforded 218 mg 28 as a light yellow oil (97% yield). ¹H NMR (CDCl₃) δ 6.52 (s, 2H), 4.64 (s, 2H), 3.87 (s, 6H), 3.84 (s, 3H). ¹³C NMR (CDCl₃) δ 153.8, 138.1, 133.3, 130.1, 104.1, 61.0, 56.4, 49.1. HRMS (EI-EMM) calc'd for [M]+m/z 239.0616. found 239.0626.

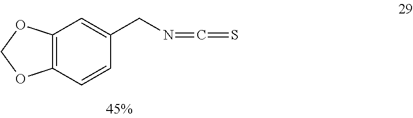 [SMILES: S=C=NCc1ccc2c(c1)OCO2]

Compound 29: 5-(isothiocyanatomethyl)benzo[d][1,3]dioxole. Compound 29 was synthesized by Method A from thiophosgene (220 μL, 332 mg, 2.885 mmols), 3,4-methylenedioxy-benzylamine (144 mg, 0.955 mmols), and NaOH (120 mg, 2.999 mmols). Silica gel chromatography (5:1 Hexane:CH₂Cl₂) and subsequent concentration afforded 84 mg 29 as a off-white solid (45% yield). ¹H NMR (CDCl₃) δ 6.79 (m, 3H), 5.98 (s, 2H), 4.60 (s, 2H). ¹³C NMR (CDCl₃) δ 148.3, 147.9, 132.5, 128.1, 120.8, 108.6, 107.8, 101.6, 48.8. HRMS (EI-EMM) calc'd for [M]+m/z 193.0198. found 193.0202.

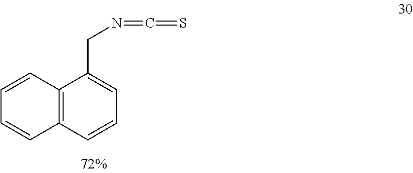 [SMILES: S=C=NCc1cccc2ccccc12]

Compound 30:1-(isothiocyanatomethyl)naphthalene. Compound 30 was synthesized by Method A from thiophosgene (220 μL, 332 mg, 2.887 mmols), 1-(methylamine)naphthalene (140 μL, 150 mg, 0.954 mmols), and NaOH (122 mg, 3.057 mmols). Silica gel chromatography (5:1 Hexane:CH₂Cl₂) and subsequent concentration afforded 137 mg 30 as a off-white solid (72% yield). ¹H NMR (CDCl₃) δ 7.86 (m, 3H), 7.60-7.41 (m, 4H), 5.07 (s, 2H). ¹³C NMR (CDCl₃) δ 133.9, 133.0, 130.6, 129.9, 129.6, 129.2, 127.2, 126.4, 126.0, 125.5, 122.7, 47.3. HRMS (EI-EMM) calc'd for [M]+m/z 199.0456. found 199.0462.

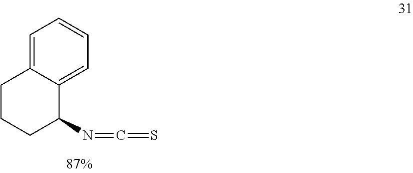 [SMILES: S=C=N[C@H]1CCCc2ccccc21]

Compound 31: (S)-1-isothiocyanato-1,2,3,4-tetrahydronaphthalene. Compound 31 was synthesized by Method A from thiophosgene (208 μL, 314 mg, 2.731 mmols), (S)-1,2,3,4-tetrahydronaphthaleneamine (138 μL, 142 mg, 0.965 mmols), and NaOH (137 mg, 3.424 mmols). Silica gel chromatography (5:1 Hexane:CH₂Cl₂) and subsequent concentration afforded 159 mg 31 as a yellow-orange oil (87% yield). ¹H NMR (CDCl₃) δ 7.33 (m, 1H), 7.21 (m, 2H), 7.10 (m, 1H), 4.90 (t, J=5.3 Hz, 1H), 2.83 (dt, J=17.0, 5.9 Hz, 1H), 2.72 (ddd, J=17.0, 7.75, 6.1 Hz, 1H), 2.08 (m, 2H), 1.96 (m, 1H), 1.81 (m, 1H). ¹³C NMR (CDCl₃) δ 136.5, 133.3, 132.0, 129.6, 128.7, 128.4, 126.6, 55.9, 30.9, 28.7, 19.4. HRMS (EI-EMM) calc'd for [M]+m/z 189.0612. found 189.0610.

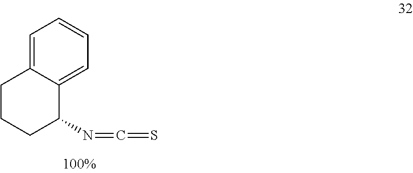 [SMILES: S=C=N[C@@H]1CCCc2ccccc21]

Compound 32: (R)-1-isothiocyanato-1,2,3,4-tetrahydronaphthalene. Compound 32 was synthesized by Method A from thiophosgene (182 μL, 274 mg, 2.387 mmols), (R)-1,2,3,4-tetrahydronaphthaleneamine (138 μL, 142 mg, 0.965 mmols), and NaOH (125 mg, 3.124 mmols). Silica gel chromatography (5:1 Hexane:CH₂Cl₂) and subsequent concentration afforded 183 mg 32 as a light-yellow oil (100% yield). ¹H NMR (CDCl₃) δ 7.32 (m, 1H), 7.20 (m, 2H), 7.10 (m, 1H), 4.89 (t, J=5.4 Hz, 1H), 2.83 (dt, J=17.0, 6.0 Hz, 1H), 2.72 (ddd, J=17.0, 7.6, 6.0 Hz, 1H), 2.08 (m, 2H), 1.96 (m, 1H), 1.81 (m, 1H). ¹³C NMR (CDCl₃) δ 136.5, 133.3, 132.0, 129.5, 128.6, 128.4, 126.6, 55.8, 30.9, 28.7, 19.4. HRMS (EI-EMM) calc'd for [M]+m/z 189.0612. found 189.0603.

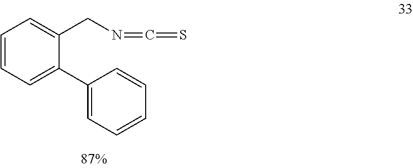 [SMILES: S=C=NCc1ccccc1-c1ccccc1]

Compound 33: 1-(isothiocyanatomethyl)-2-phenylbenzene. Compound 33 was synthesized by Method A from thiophosgene (196 μL, 296 mg, 2.574 mmols), 2-phenylbenzylamine hydrochloride (213 mg, 0.969 mmols), DIEA (250 μL, 186 mg, 1.441 mmols), and NaOH (134 mg, 3.349 mmols). Silica gel chromatography (5:1 Hexane: CH₂Cl₂) and subsequent concentration afforded 189 mg 33 as a deep-orange oil (87% yield). ¹H NMR (CDCl₃) δ 7.49-7.45 (m, 1H), 7.43-7.32 (m, 5H), 7.27-7.23 (m, 3H), 4.55 (s, 2H). ¹³C NMR (CDCl₃) δ 141.4, 139.8, 132.0, 131.8, 130.4, 129.09, 128.6, 128.6, 128.4, 128.2, 127.8, 47.1. HRMS (EI-EMM) calc'd for [M]+m/z 225.0612. found 225.0618.

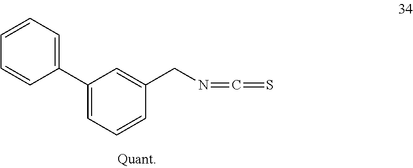 [SMILES: S=C=NCc1cccc(-c2ccccc2)c1]

Compound 34: 1-(isothiocyanatomethyl)-3-phenylbenzene. Compound 34 was synthesized by Method A from thiophosgene (196 μL, 296 mg, 2.574 mmols), 3-phenylbenzylamine hydrochloride (220 mg, 1.001 mmols), DIEA (250 μL, 186 mg, 1.441 mmols), and NaOH (134 mg, 3.349 mmols). Silica gel chromatography (5:1 Hexane: CH₂Cl₂) and subsequent concentration afforded 34 as a deep-orange oil in quantitative yield. ¹H NMR (CDCl₃) δ 7.56-7.48 (m, 3H), 7.47-7.37 (m, 4H), 7.33 (m, 1H), 7.22 (m, 1H), 4.66 (s, 2H). ¹³C NMR (CDCl₃) δ 142.1, 140.4, 134.9, 132.6, 129.5, 129.0, 127.8, 127.2, 127.2, 125.7, 125.6, 48.8. HRMS (EI-EMM) calc'd for [M]+m/z 225.0612. found 225.0609.

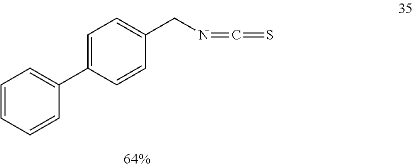 [SMILES: S=C=NCc1ccc(-c2ccccc2)cc1]

Compound 35: 1-(isothiocyanatomethyl)-4-phenylbenzene. Compound 35 was synthesized by Method A from thiophosgene (220 μL, 332 mg, 2.887 mmols), 4-phenyl-benzylamine (177 mg, 0.966 mmols), and NaOH (122 mg, 3.069 mmols). Silica gel chromatography (5:1 Hexane:CH₂Cl₂) and subsequent concentration afforded 137 mg 35 as a off-white solid (64% yield). ¹H NMR (CDCl₃) δ 7.58 (m, 4H), 7.43 (m, 2H), 7.35 (m, 3H), 4.71 (s, 2H). ¹³C NMR (CDCl₃) δ 141.5, 140.5, 133.3, 132.6, 129.0, 127.8, 127.8, 127.5, 127.2, 48.6. HRMS (EI-EMM) calc'd for [M]+m/z 225.0612. found 225.0622.

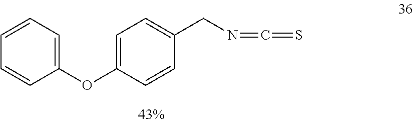 [SMILES: S=C=NCc1ccc(Oc2ccccc2)cc1]

Compound 36: 1-(isothiocyanatomethyl)-4-phenoxybenzene. Compound 36 was synthesized by Method A from thiophosgene (196 μL, 296 mg, 2.574 mmols), 4-phenoxybenzylamine (198 mg, 0.994 mmols), and NaOH (125 mg, 3.124 mmols). Silica gel chromatography (5:1 Hexane:CH₂Cl₂) and subsequent concentration afforded 104 mg 36 as a light-orange oil (43% yield). ¹H NMR (CDCl₃) δ 7.34 (m, 2H), 7.25 (m, 2H), 7.12 (tt, J=7.4, 1.1 Hz, 1H), 7.02-6.98 (m, 4H), 4.65 (s, 2H). ¹³C NMR (CDCl₃) δ 157.7, 156.9, 132.5, 130.0, 129.0, 128.7, 123.9, 119.3, 119.1, 48.4. HRMS (EI-EMM) calc'd for [M]+m/z 241.0561. found 241.0550.

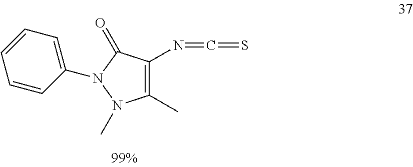 [SMILES: Cc1c(N=C=S)c(=O)n(-c2ccccc2)n1C]

Compound 37: 4-isothiocyanato-2,3-dimethyl-1-phenyl-1,2-dihydropyrazol-5-one. Compound 37 was synthesized by Method A from thiophosgene (208 μL, 314 mg, 2.727 mmols), 4-amino-antipyrine (196 mg, 0.964 mmols), and NaOH (110 mg, 2.749 mmols). Silica gel chromatography (EtOAc) and subsequent concentration afforded 235 mg 37 as a light-yellow solid (99% yield). ¹H NMR (CDCl₃) δ 7.45 (t, J=7.7 Hz, 2H), 7.32 (m, 3H), 3.10 (s, 3H), 2.27 (s, 3H). ¹³C NMR (CDCl₃) δ 160.9, 147.9, 142.9, 134.1, 129.3, 127.5, 124.5, 103.6, 35.7, 10.9. HRMS (EI-EMM) calc'd for [M]+m/z 245.0623. found 245.0635).

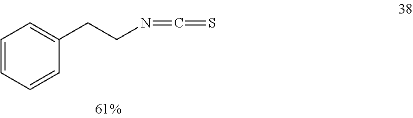 [SMILES: S=C=NCCc1ccccc1]

Compound 38: 1-(2-isothiocyanatoethyl)benzene (PEITC). Compound 38 was synthesized by Method A from thiophosgene (196 μL, 296 mg, 2.574 mmols), 2-pheynlethylamine (122 μL, 117 mg, 0.966 mmols), and NaOH (131 mg, 3.274 mmols). Silica gel chromatography (5:1 Hexane:CH₂Cl₂) and subsequent concentration afforded 97 mg 38 as a light-orange oil (61% yield). ¹H NMR (CDCl₃) δ 7.33 (m, 2H), 7.26 (m, 1H), 7.21 (m, 2H), 3.70 (t, J=7.0 Hz, 2H), 2.97 (t, J=7.0 Hz, 2H). ¹³C NMR (CDCl₃) δ 137.1, 131.0, 128.9, 127.3, 46.5, 36.6. HRMS (EI-EMM) calc'd for [M]+m/z 163.0456. found 163.0463.

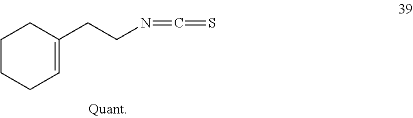 [SMILES: S=C=NCCC1=CCCCC1]

Compound 39: 1-(2-isothiocyanatoethyl)cyclohex-1-ene. Compound 39 was synthesized by Method A from thiophosgene (206 μL, 311 mg, 2.705 mmols), 2-(1-cyclohexenyl)-ethylamine (135 μL, 121 mg, 0.966 mmols), and NaOH (140 mg, 3.499 mmols). Silica gel chromatography (5:1 Hexane:CH₂Cl₂) and subsequent concentration afforded 39 as an orange oil in quantitative yield. ¹H NMR (CDCl₃) δ 5.54 (m, 1H), 3.53 (t, J=6.7 Hz, 2H), 2.31 (t, J=6.8 Hz, 2H), 2.01 (m, 2H), 1.91 (m, 2H), 1.67-1.60 (m, 2H), 1.60-1.52 (m, 2H). ¹³C NMR (CDCl₃) δ 132.8, 131.0, 125.6, 43.8, 38.7, 27.9, 25.4, 22.9, 22.3. HRMS (EI-EMM) calc'd for [M]+m/z 163.0769. found 163.0771.

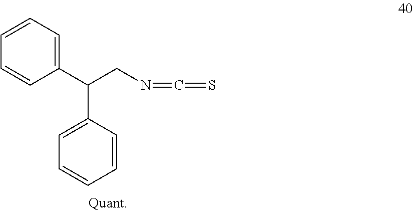 [SMILES: S=C=NCC(c1ccccc1)c1ccccc1]

Compound 40: 2-isothiocyanato-1,1-diphenylethane. Compound 40 was synthesized by Method A from thiophosgene (220 μL, 332 mg, 2.887 mmols), 2,2-diphenylethylamine (192 mg, 0.974 mmols), and NaOH (123 mg, 3.061 mmols). Silica gel chromatography (5:1 Hexane:CH₂Cl₂) and subsequent concentration afforded 40 as a light-yellow oil in quantitative yield. ¹H NMR (CDCl₃) δ 7.30 (m, 4H), 7.25-7.16 (m, 6H), 4.31 (t, J=7.6, 1H), 3.99 (d, J=7.5 Hz, 2H). ¹³C NMR (CDCl₃) δ 140.4, 131.9, 128.9, 128.0, 127.4, 51.4, 49.4. HRMS (EI-EMM) calc'd for [M]+m/z 239.0769. found 239.0763.

Calcein-AM and Cell Titer-Glo Cytotoxicity Assays. All cell lines except NmuMG were maintained in RPMI medium 1640 supplemented with 10% (wt/vol) FBS and penicillin-streptomycin (PS) (100 units/mL and 100 μg/mL). NmuMG cells were maintained in DMEM supplemented with 10% wt/vol FBS, 10 μg/mL insulin, and penicillin/streptomycin (PS) (100 units/mL and 100 μg/mL, respectively). Cells were harvested by trypsinization using 0.25% trypsin and 0.1% EDTA and then counted in a hemocytometer in duplicate with better than 10% agreement in field counts. Cells were plated at a density of 10,000-15,000 cells per well of each 96-well black tissue culture treated microtiter plate. Cells were grown for 1 h at 37° C., with 5% CO₂/95% air in a humidified incubator to allow cell attachment to occur before compound addition. Library members were stored at −20° C. under desiccating conditions before the assay. Library member stocks (100×) were prepared in 96-well V-bottom polypropylene microtiter plates. Five serial 1:2 dilutions were made with anhydrous DMSO at 100× the final concentration used in the assay. The library member-containing plates were diluted 1:10 with complete cell culture medium. The 10× stocks (10 μL) were added to the attached cells by using a Biomek FX liquid handler (Beckman Coulter). Library member stocks (10 μL) were added to 90 μL of cells in each plate to ensure full mixing of stocks with culture media by using a Biomek FK liquid handler with 96-well head. Cells were incubated with the library members for 72 h before fluorescence reading. Test plates were removed from the incubator and washed once in sterile PBS to remove serum containing calcium esterases. Calcein AM (acetoxymethyl ester) reagent (30 μL, 1 M) was added and the cells were incubated for 30 min at 37° C. Plates were read for emission by using a fluorescein filter (excitation 485 nm, emission 535 nm). An equal volume (30 μL) of cell titer-glo reagent (Promega Corporation, Inc.) was added and incubated for 10 min at room temperature with gentle agitation to lyse the cells. Each plate was re-read for luminescence to confirm the inhibition observed in the fluorescent Calcein AM assay.

MTT Cytotoxicity Assay. HT-29 cell lines were maintained in RPMI medium 1640 supplemented with 10% (wt/vol) FBS and 1% penicillin-streptomycin (PS) (100 units/mL and 100 μg/mL). Cells were harvested by trypsinization using 0.25% trypsin and 0.1% EDTA and plated at a density of 2,000-5,000 cells per well of each 96-well microtiter plate. Cells were grown for 24 h at 37° C., with 5% CO₂/95% air in a humidified incubator to allow cell attachment to occur before compound addition. Library members were stored at −20° C. under desiccating conditions before the assay. Library member stocks (1000×) were prepared in falcon tubes (BD Biosciences). Five dilutions were made with DMSO at 1000× the final concentration used in the assay. The 1000× stocks (3 μL) were diluted with complete culture medium (3 mL). Each plate contained twelve replicates of cells treated with 0.1% DMSO in complete cell culture medium that served as an individual negative control. Library member stocks (1×, 200 μL) were added to aspirated cells and replicated twelve times on each plate. Cells were incubated with the library members for 24 h before optical density reading. Test plates were removed from the incubator and washed once in sterile PBS to remove serum containing library members. Freshly-made 10×MTT solution in sterile PBS (5 mg/mL) was diluted 1:10 in serum-free culture medium and was sterilized using a 0.2 μm filter. Diluted MTT solution (1×, 50 μL) was added to each well and the plates were incubated in the dark for 2 h. Test plates were centrifuged at 1800×g for 5 min at 4° C. using a Beckman GPR centrifuge. MTT solution was removed by aspiration and 100 μL DMSO was added to each well to lyse cells and solubilize formazan crystals. Test plates were incubated in the dark at room temperature for 10 min. Plates were read for optical density at 570 nm.

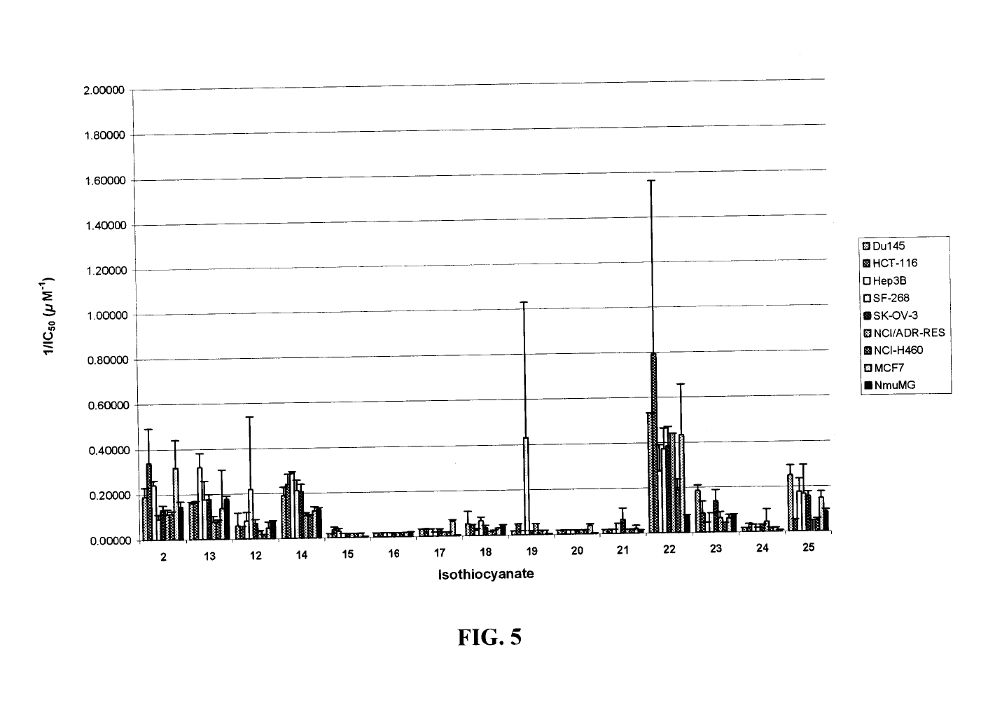

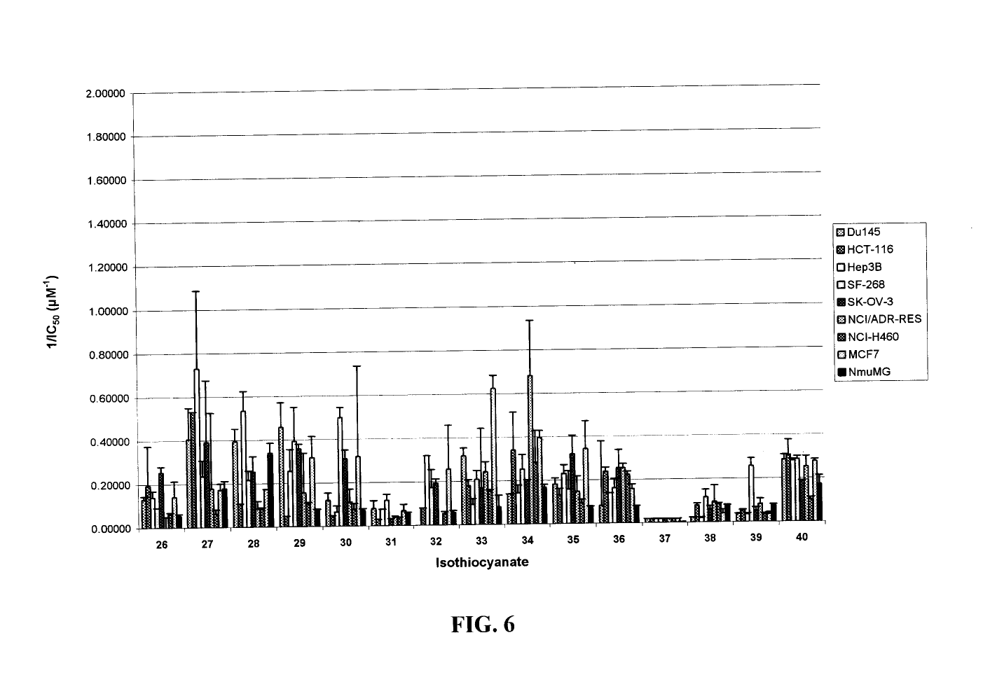

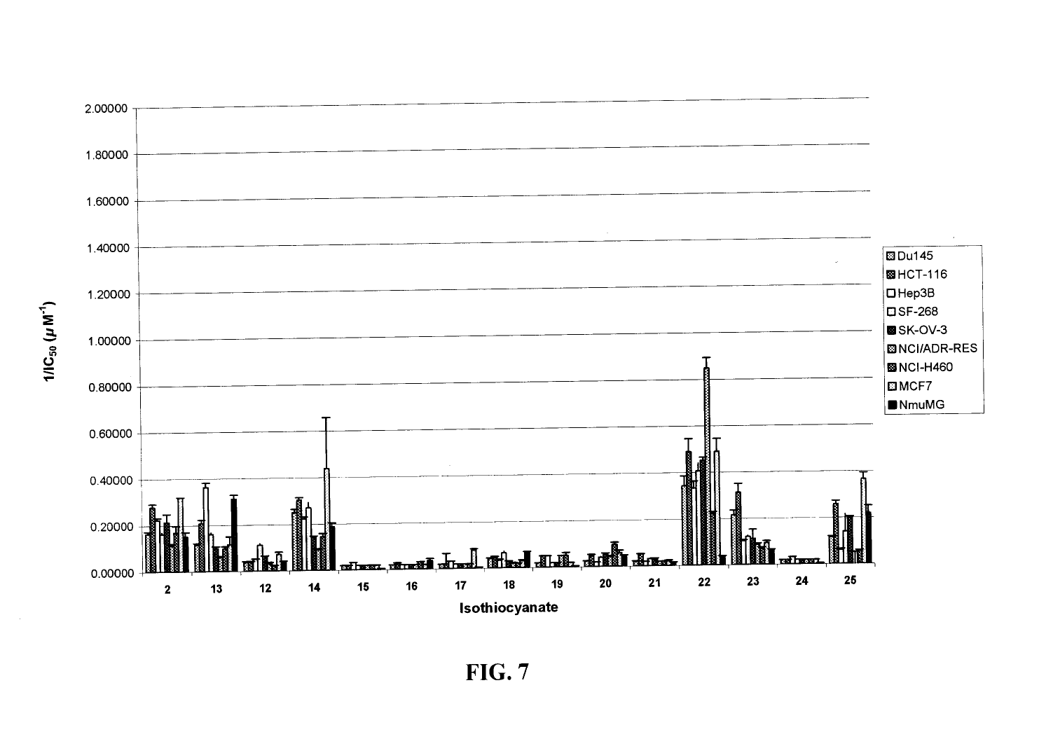

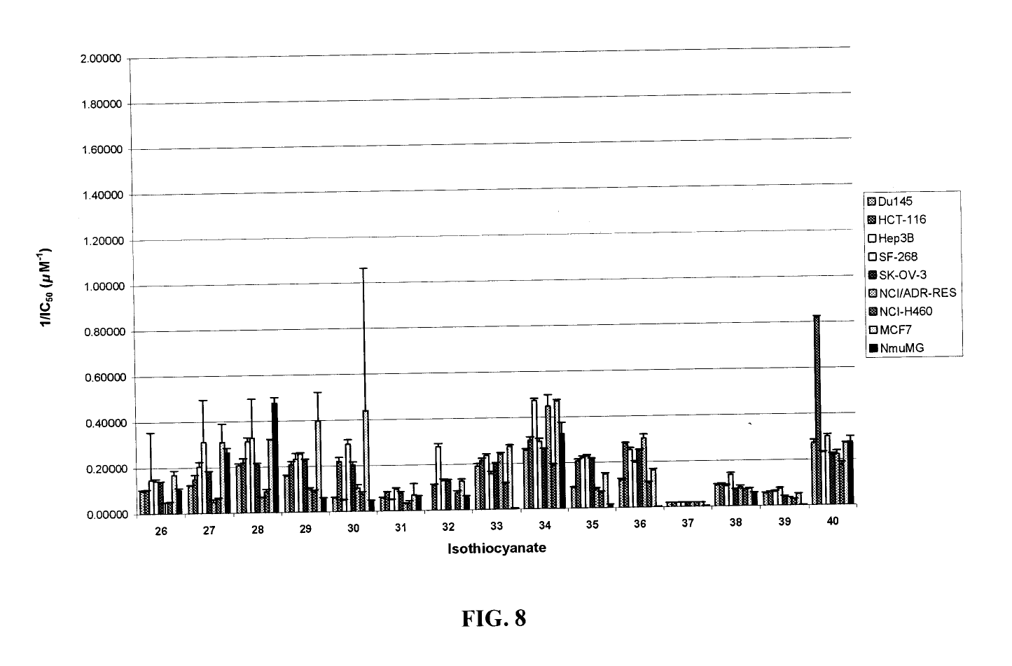

IC₅₀ Calculations. For each library member, at least three dose-response experiments were conducted on separate plates. For each experiment, percent inhibition values at each concentration were expressed as a percentage of the maximum emission signal observed for a 0 μM control. To calculate IC₅₀, percent inhibitions were plotted as a function of log [concentration] and then fit to a four-parameter logistic model that allowed for a variable Hill slope by using XLFIT 4.1 (ID Business Solutions, Emeryville, Calif.). The results are presented in Tables 1-6 and in FIGS. 5-8.

TABLE 1

| | | 2 | | 13 | | 12 | | 14 | | 15 | |
|---|---|---|---|---|---|---|---|---|---|---|---|
| Cell Line | Assay | IC₅₀ | SE | IC₅₀ | SE | IC₅₀ | SE | IC₅₀ | SE | IC₅₀ | SE |
| Du145 | Calcein AM | 5.25 | 0.91 | 6.25 | 0.30 | 16.68 | 8.05 | 5.24 | 0.85 | >50.00 | N/A |
| | Cell Titer-Glo | 6.10 | 0.31 | 8.62 | 0.43 | 25.78 | 2.13 | 4.00 | 0.22 | >50.00 | N/A |
| HCT-116 | Calcein AM | 2.94 | 0.91 | 6.13 | 0.27 | 22.57 | 4.91 | 4.16 | 0.66 | 27.55 | 7.02 |
| | Cell Titer-Glo | 3.58 | 0.15 | 4.81 | 0.33 | 24.65 | 1.21 | 3.29 | 0.12 | >50.00 | N/A |
| Hep3B | Calcein AM | 4.13 | 0.31 | <3.13 | 0.51 | 12.45 | 4.02 | 3.46 | 0.08 | 32.77 | 8.32 |
| | Cell Titer-Glo | 4.48 | 0.16 | 2.75 | 0.14 | 19.36 | 0.71 | 4.48 | 0.16 | >30.00 | N/A |
| SF-268 | Calcein AM | 11.04 | 2.01 | 5.69 | 1.82 | 4.54 | 2.69 | 4.72 | 0.85 | >50.00 | N/A |
| | Cell Titer-Glo | 6.16 | 0.24 | 6.22 | 0.26 | 8.89 | 0.44 | 3.70 | 0.30 | >50.00 | N/A |
| SK-OV-3 | Calcein AM | 7.60 | 1.04 | 5.65 | 0.66 | 14.55 | 2.89 | 4.84 | 0.67 | >50.00 | N/A |
| | Cell Titer-Glo | 4.67 | 0.65 | 9.90 | 0.66 | 15.99 | 0.84 | 6.99 | 0.29 | >50.00 | N/A |
| NCI/ADR- | Calcein AM | 8.89 | 1.42 | 13.12 | 3.50 | >30.00 | 3.31 | 9.99 | 1.18 | >50.00 | N/A |
| RES | Cell Titer-Glo | 8.98 | 0.67 | 16.49 | 1.39 | >30.00 | 3.73 | 11.11 | 0.60 | >50.00 | N/A |
| NCI-H460 | Calcein AM | 8.74 | 0.79 | 12.96 | 1.34 | >50.00 | N/A | 10.01 | 0.47 | >50.00 | N/A |
| | Cell Titer-Glo | 5.94 | 0.86 | 10.14 | 0.81 | 45.29 | 4.91 | 6.87 | 0.60 | >50.00 | N/A |
| MCF7 | Calcein AM | 3.14 | 0.87 | 7.28 | 4.03 | 21.02 | 7.57 | 8.36 | 1.22 | >50.00 | N/A |
| | Cell Titer-Glo | <3.13 | 9.41 | 8.59 | 1.93 | 13.99 | 1.70 | 2.28* | 0.76 | >50.00 | N/A |
| NmuMG | Calcein AM | 6.86 | 0.94 | 5.61 | 0.43 | <12.50 | 0.00 | 7.90 | 0.47 | >200.00 | N/A |
| | Cell Titer-Glo | 6.59 | 0.70 | 3.20 | 0.16 | 23.52 | N/A | 5.32 | 0.43 | >200.00 | N/A |
| HT-29 | MTT | 45.21 | 0.93 | 46.39 | 1.70 | 37.71 | 0.74 | 39.15 | 0.83 | >50.00 | N/A |

| | | | | | |
|---|---|---|---|---|---|
| Cancerous Average | 5.26 | 7.61 | 21.99 | 5.32 | >50.00 |

TABLE 2

| | | 16 | | 17 | | 18 | | 19 | | 20 | |
|---|---|---|---|---|---|---|---|---|---|---|---|
| Cell Line | Assay | IC₅₀ | SE | IC₅₀ | SE | IC₅₀ | SE | IC₅₀ | SE | IC₅₀ | SE |
| Du145 | Calcein AM | >50.00 | N/A | 29.69 | N/A | 18.71 | 9.53 | >50.00 | N/A | >50.00 | N/A |
| | Cell Titer-Glo | >50.00 | N/A | >50.00 | N/A | 24.37 | 1.51 | >50.00 | N/A | 41.37 | 2.20 |
| HCT-116 | Calcein AM | >50.00 | N/A | >50.00 | 21.34 | 21.91 | 2.27 | >20.00 | N/A | >50.00 | N/A |
| | Cell Titer-Glo | 35.00 | N/A | >50.00 | 34.94 | 20.93 | 1.30 | >20.00 | N/A | 21.64 | 2.75 |
| Hep3B | Calcein AM | >50.00 | N/A | >30.00 | N/A | 42.08 | 9.85 | >50.00 | N/A | >50.00 | N/A |
| | Cell Titer-Glo | >50.00 | N/A | >30.00 | N/A | 29.33 | 3.18 | >20.00 | N/A | >50.00 | N/A |
| SF-268 | Calcein AM | >50.00 | N/A | >50.00 | N/A | 15.07 | 2.78 | 2.32 | 1.35 | >50.00 | N/A |
| | Cell Titer-Glo | >50.00 | N/A | >50.00 | N/A | 15.36 | 1.46 | >50.00 | N/A | 24.90 | 1.45 |
| SK-OV-3 | Calcein AM | >50.00 | N/A | >30.00 | N/A | 29.08 | 7.47 | >50.00 | N/A | >50.00 | N/A |
| | Cell Titer-Glo | >50.00 | N/A | >50.00 | N/A | 33.70 | 1.46 | >50.00 | N/A | 19.59 | 2.63 |
| NCI/ADR- | Calcein AM | >50.00 | N/A | >50.00 | N/A | >50.00 | N/A | >20.00 | N/A | >50.00 | N/A |
| RES | Cell Titer-Glo | >50.00 | N/A | >50.00 | N/A | >50.00 | 5.53 | >20.00 | N/A | 23.11 | 1.89 |
| NCI-H460 | Calcein AM | >50.00 | N/A | >50.00 | N/A | 48.14 | 6.95 | >50.00 | N/A | >50.00 | N/A |
| | Cell Titer-Glo | 30.29 | N/A | >50.00 | N/A | >50.00 | N/A | >20.00 | 4.13 | 10.44 | 0.84 |
| MCF7 | Calcein AM | >50.00 | N/A | 15.12 | 1.33 | >30.00 | N/A | >50.00 | N/A | 26.68 | 4.89 |
| | Cell Titer-Glo | >50.00 | N/A | 12.56 | 0.78 | 33.31 | 3.66 | >50.00 | N/A | 17.08 | 2.72 |
| NmuMG | Calcein AM | 49.44 | 4.50 | >200.00 | N/A | 22.49 | 1.56 | >200.00 | N/A | 182.75 | N/A |
| | Cell Titer-Glo | 26.80 | 4.97 | >200.00 | N/A | 14.76 | 0.43 | >200.00 | N/A | 21.45 | 1.19 |
| HT-29 | MTT | >50.00 | N/A | >50.00 | N/A | >50.00 | 2.73 | >50.00 | N/A | >50.00 | N/A |

| | | | | | |
|---|---|---|---|---|---|
| Cancerous Average | >50.00 | >50.00 | 29.31 | >50.00 | >50.00 |

TABLE 3

| | | 21 | | 22 | | 23 | | 24 | | 25 | |
|---|---|---|---|---|---|---|---|---|---|---|---|
| Cell Line | Assay | IC₅₀ | SE | IC₅₀ | SE | IC₅₀ | SE | IC₅₀ | SE | IC₅₀ | SE |
| Du145 | Calcein AM | >50.00 | N/A | <1.88 | N/A | 5.37 | 0.62 | >50.00 | N/A | 3.99 | 0.60 |
| | Cell Titer-Glo | 48.02 | 3.65 | 2.91 | 0.32 | 4.64 | 0.41 | >50.00 | N/A | 8.64 | 0.30 |
| HCT-116 | Calcein AM | >50.00 | N/A | 1.26* | 0.62 | 11.54 | 4.29 | >50.00 | 23.19 | 19.56 | 1.42 |
| | Cell Titer-Glo | 19.36 | 0.82 | 2.04 | 0.21 | 3.19* | 0.34 | >50.00 | N/A | 3.88 | 0.21 |
| Hep3B | Calcein AM | >50.00 | N/A | 3.64 | 1.08 | 22.46 | N/A | >30.00 | N/A | 5.67 | 1.42 |
| | Cell Titer-Glo | >50.00 | N/A | 3.00 | 0.25 | 9.70 | 0.37 | >30.00 | N/A | 16.28 | 0.91 |
| SF-268 | Calcein AM | 21.52 | 44.50 | 2.68 | 0.53 | 11.50 | N/A | >50.00 | N/A | 6.00 | 2.63 |
| | Cell Titer-Glo | 32.13 | 3.35 | 2.46 | 0.19 | 8.27 | 0.30 | >50.00 | N/A | 7.11 | 2.43 |
| SK-OV-3 | Calcein AM | 15.89 | 7.09 | 2.60 | 0.48 | 7.18 | 1.89 | >30.00 | N/A | 6.32 | 0.71 |
| | Cell Titer-Glo | 31.38 | 2.21 | 2.21 | 0.06 | 8.86 | 2.33 | >50.00 | N/A | 5.01 | 0.13 |
| NCI/ADR- | Calcein AM | >50.00 | N/A | 2.27 | N/A | 15.52 | 4.59 | 21.57 | 11.71 | >20.00 | N/A |
| RES | Cell Titer-Glo | >50.00 | N/A | 1.18 | 0.06 | 11.48 | 0.77 | >50.00 | N/A | 20.13* | 0.88 |
| NCI-H460 | Calcein AM | >50.00 | N/A | 5.18 | 0.99 | 22.95 | N/A | >50.00 | N/A | 19.41 | 2.45 |
| | Cell Titer-Glo | >50.00 | N/A | 4.51 | 0.18 | 14.46 | 1.19 | >50.00 | N/A | 17.34 | 0.70 |
| MCF7 | Calcein AM | 45.83 | 18.00 | 2.31 | 0.79 | 16.00 | 3.28 | >50.00 | N/A | 6.76 | 1.15 |
| | Cell Titer-Glo | 45.35 | 3.51 | 2.04 | 0.21 | 10.56 | 0.84 | >50.00 | N/A | 2.73* | 0.19 |
| NmuMG | Calcein AM | 71.91 | 8.50 | <12.50 | N/A | <12.50 | N/A | 150.68 | 29.67 | 11.34 | 1.17 |
| | Cell Titer-Glo | 65.67 | 5.06 | 26.04 | 1.15 | 15.44 | 0.25 | >200.00 | N/A | 4.60 | 0.62 |
| HT-29 | MTT | >50.00 | N/A | 21.33 | 0.65 | 31.45 | 1.03 | >50.00 | N/A | 26.94 | 0.64 |

| | | | | | |
|---|---|---|---|---|---|
| Cancerous Average | 35.25 | 2.54 | 9.47 | >50.00 | 10.14 |

TABLE 4

| | | 26 | | 27 | | 28 | | 29 | | 30 | |
|---|---|---|---|---|---|---|---|---|---|---|---|
| Cell Line | Assay | IC₅₀ | SE | IC₅₀ | SE | IC₅₀ | SE | IC₅₀ | SE | IC₅₀ | SE |
| Du145 | Calcein AM | 7.67 | 0.79 | 2.46 | 0.64 | 2.54 | 0.33 | 2.18 | 0.43 | 8.31 | 1.79 |
| | Cell Titer-Glo | 10.09 | 0.49 | 8.24 | 0.21 | 4.80 | 0.17 | 6.24 | 0.17 | 16.76 | 1.06 |
| HCT-116 | Calcein AM | 5.16 | 2.49 | <1.88 | N/A | 9.23 | N/A | >20.00 | N/A | >20.00 | N/A |
| | Cell Titer-Glo | 9.80 | 0.36 | 6.72 | 0.78 | 4.55 | 0.35 | 4.77 | 0.30 | 4.55 | 0.34 |
| Hep3B | Calcein AM | 7.16 | 1.25 | 1.37 | 0.45 | 1.87 | 0.27 | 3.90 | 1.09 | 15.19 | 4.60 |
| | Cell Titer-Glo | 6.80 | 3.99 | 4.87 | 0.43 | 3.21 | 0.17 | 4.30 | 0.42 | 19.79 | 1.02 |
| SF-268 | Calcein AM | 10.91 | N/A | 4.26 | 1.01 | 4.61 | 0.75 | 2.55 | 0.73 | 2.00 | 0.17 |
| | Cell Titer-Glo | 6.96 | 0.40 | 3.21 | 1.19 | 3.05 | 1.04 | 3.92 | 0.09 | 3.39 | 0.21 |
| SK-OV-3 | Calcein AM | 3.94 | 0.38 | 2.55 | 1.07 | 3.93 | 0.85 | 2.79* | 0.16 | 3.22 | 0.37 |
| | Cell Titer-Glo | 7.17 | 0.12 | 5.47 | 0.06 | 4.75 | 0.16 | 4.44 | 0.14 | 4.89 | 0.29 |
| NCI/ADR- | Calcein AM | >20.00 | N/A | 5.61 | 3.71 | 12.01 | 3.59 | 6.39 | 3.43 | 8.93 | 3.06 |
| RES | Cell Titer-Glo | 22.27* | 1.61 | 20.83 | 4.08 | 15.20 | 0.71 | 9.77 | 0.53 | 9.71 | 1.21 |
| NCI-H460 | Calcein AM | 15.98 | 1.41 | 15.78 | 3.53 | 11.94 | 0.78 | 9.59 | 0.71 | 12.64 | 3.11 |
| | Cell Titer-Glo | >20.00 | 0.80 | 16.09 | 1.13 | 10.81 | 1.11 | 11.47 | 1.14 | 13.27 | 1.77 |
| MCF7 | Calcein AM | 7.08 | 2.41 | 5.81 | 0.82 | 5.75 | N/A | 3.15 | 0.74 | <3.13 | 1.77 |
| | Cell Titer-Glo | 5.92 | 0.61 | 3.22* | 0.66 | 3.15 | 0.04 | 2.51* | 0.60 | 2.27* | 1.33 |
| NmuMG | Calcein AM | 18.27 | 2.32 | 5.59 | 0.90 | 2.94 | 0.35 | <12.50 | N/A | 13.85 | 1.52 |
| | Cell Titer-Glo | 9.77 | 0.73 | 3.78 | 0.26 | 2.09 | 0.10 | 16.99 | 1.06 | 21.45 | 1.19 |
| HT-29 | MTT | 47.40 | 1.19 | ND | ND | 17.02 | 0.81 | 30.34 | 0.63 | >50.00 | N/A |

| | | | | | |
|---|---|---|---|---|---|
| Cancerous Average | 10.67 | 6.37 | 6.53 | 5.77 | 9.16 |

TABLE 5

| | | 31 | | 32 | | 33 | | 34 | | 35 | |
|---|---|---|---|---|---|---|---|---|---|---|---|
| Cell Line | Assay | IC₅₀ | SE | IC₅₀ | SE | IC₅₀ | SE | IC₅₀ | SE | IC₅₀ | SE |
| Du145 | Calcein AM | 12.43 | 3.84 | 5.27 | 0.77 | 3.16 | 0.33 | 7.11 | N/A | 5.49 | 0.74 |
| | Cell Titer-Glo | 17.37 | 0.93 | 8.66 | 0.45 | 5.25 | 0.30 | 3.87 | 0.08 | 10.95 | 0.39 |
| HCT-116 | Calcein AM | 32.97 | N/A | 12.48 | N/A | 5.56 | 0.76 | 2.93 | 0.99 | 7.54 | 1.39 |
| | Cell Titer-Glo | 12.19 | 0.46 | 9.05 | 0.39 | 4.64 | 0.22 | 3.33 | 0.16 | 4.83 | 0.21 |
| Hep3B | Calcein AM | 13.07 | N/A | <3.13 | 6.93 | 10.63 | 2.34 | 6.90 | 1.35 | 4.33 | 0.64 |
| | Cell Titer-Glo | 20.09 | 0.53 | 3.59 | 0.16 | 4.24 | 0.15 | 2.11 | 0.04 | 4.51 | 0.14 |
| SF-268 | Calcein AM | 8.51 | 1.64 | 5.75 | 1.85 | 4.79 | 0.79 | 3.95 | 0.82 | 6.26 | 2.15 |
| | Cell Titer-Glo | 10.52 | 0.69 | 7.73 | 0.46 | 6.39 | 0.39 | 3.39 | 0.15 | 4.40 | 0.12 |
| SK-OV-3 | Calcein AM | >30.00 | N/A | 5.18 | 0.49 | 5.93 | 3.67 | 4.92 | ND | <3.13 | 0.67 |
| | Cell Titer-Glo | 12.68 | 0.87 | 7.74 | 0.30 | 5.01 | 0.15 | 3.83 | 0.07 | 4.65 | 0.09 |
| NCI/ADR- | Calcein AM | 25.08 | 2.88 | ND | ND | 4.12 | 0.66 | 1.47* | 0.40 | 6.73 | 2.16 |
| RES | Cell Titer-Glo | 30.87 | 2.81 | ND | ND | 4.11 | 0.09 | 2.23 | 0.22 | 12.09 | 0.84 |
| NCI-H460 | Calcein AM | 25.41 | 0.14 | 18.73 | 2.74 | 6.44 | 0.24 | 3.53 | 1.20 | 10.47 | 1.56 |
| | Cell Titer-Glo | 31.63 | 8.84 | 13.30 | 1.42 | 8.95 | 0.47 | 5.29 | 0.22 | 14.95 | 1.54 |
| MCF7 | Calcein AM | 14.47 | 4.35 | 3.88 | 1.70 | 1.60* | 0.14 | 2.52 | 0.19 | 2.91* | 0.79 |
| | Cell Titer-Glo | 14.44 | 6.05 | 7.83 | 0.46 | 3.63 | 0.09 | 2.13 | 0.03 | 6.72 | 0.25 |
| NmuMG | Calcein AM | 18.64 | 2.38 | 17.24 | 2.45 | <12.50 | 5.06 | 5.95 | 0.46 | <12.50 | N/A |
| | Cell Titer-Glo | 15.44 | 0.25 | 16.99 | 1.06 | 180.10 | N/A | 3.06 | 0.41 | 65.67 | 5.06 |
| HT-29 | MTT | >50.00 | 1.31 | 20.36 | N/A | 39.85 | 1.00 | 28.82 | 0.52 | >50.00 | N/A |

| | | | | | |
|---|---|---|---|---|---|
| Cancerous Average | 17.23 | 8.27 | 4.96 | 3.27 | 7.89 |

TABLE 6

| | | 36 | | 37 | | 38 | | 39 | | 40 | |
|---|---|---|---|---|---|---|---|---|---|---|---|
| Cell Line | Assay | IC IC₅₀50 | SE | IC₅₀ | SE | IC₅₀ | SE | IC₅₀ | SE | IC₅₀ | SE |
| Du145 | Calcein AM | 12.16 | 9.52 | >50.00 | N/A | 43.05 | N/A | 25.52 | 0.21 | 3.51 | 0.27 |
| | Cell Titer-Glo | 8.24 | 0.43 | >50.00 | N/A | 10.86 | 0.63 | 18.58 | 1.34 | 3.65 | 0.17 |
| HCT-116 | Calcein AM | 4.19 | 0.31 | >50.00 | N/A | 12.73 | 1.38 | 18.98 | 1.55 | 3.26 | 0.61 |
| | Cell Titer-Glo | 3.55 | 0.04 | >50.00 | N/A | 10.27 | 0.39 | 16.39 | 0.51 | 1.22* | 0.01 |
| Hep3B | Calcein AM | 7.17 | N/A | >50.00 | N/A | 46.25 | 5.63 | 28.46 | 3.71 | 3.58 | 0.10 |
| | Cell Titer-Glo | 3.95 | 0.14 | >50.00 | N/A | 11.12 | 0.54 | 16.11 | 0.97 | 4.30 | 0.05 |
| SF-268 | Calcein AM | 6.17 | 1.31 | >50.00 | N/A | 8.66 | 2.01 | 3.89 | 0.48 | 3.49 | 0.16 |
| | Cell Titer-Glo | 5.06 | 0.18 | >50.00 | N/A | 7.20 | 0.39 | 13.58 | 1.01 | 3.33 | 0.11 |
| SK-OV-3 | Calcein AM | 3.93 | 0.97 | >50.00 | N/A | 16.67 | 3.49 | 19.51 | 4.78 | 5.26 | N/A |
| | Cell Titer-Glo | 3.99 | 0.07 | >50.00 | N/A | 13.51 | 0.48 | 24.05 | 0.89 | 4.52 | 0.14 |
| NCI/ADR- | Calcein AM | 3.93 | 0.27 | >50.00 | N/A | 10.54 | 4.66 | 12.19 | 2.93 | 3.95 | 0.63 |
| RES | Cell Titer-Glo | 3.30 | 0.18 | >50.00 | N/A | 12.14 | 1.13 | 26.95 | N/A | 4.47 | 0.30 |
| NCI-H460 | Calcein AM | 4.47 | 0.28 | >50.00 | N/A | 12.10 | 0.74 | 28.63 | 3.41 | 9.91 | 1.00 |
| | Cell Titer-Glo | 9.12 | 0.36 | >50.00 | N/A | 13.67 | 0.45 | 36.35 | 4.18 | 5.64 | 0.41 |
| MCF7 | Calcein AM | 6.34 | 0.76 | >50.00 | N/A | 23.68 | 6.93 | 27.50 | 4.67 | 3.62 | 0.13 |
| | Cell Titer-Glo | 6.16 | 0.24 | >50.00 | N/A | 13.07 | 0.62 | 19.75 | 1.18 | 3.68 | 0.04 |
| NmuMG | Calcein AM | <12.50 | 0.01 | >200.00 | N/A | <12.50 | N/A | 13.03 | 0.73 | 5.81 | 1.12 |
| | Cell Titer-Glo | 186.59 | N/A | >200.00 | N/A | 17.74 | 1.02 | >200.00 | N/A | 3.62 | 0.30 |
| HT-29 | MTT | 37.64 | 0.75 | >50.00 | 9.51 | 33.18 | 1.00 | 45.28 | 0.95 | 22.95 | 0.82 |

| | | | | | |
|---|---|---|---|---|---|
| Cancerous Average | 4.84 | >50.00 | 11.48 | 18.32 | 4.11 |

Syntheses of D,L-Sulforaphane and Erysolin. The syntheses of D,L-sulforaphane and erysolin were carried out as highlighted in Reaction Scheme 1, supra. This overall procedure was modified from previously-reported work by Vermeulen et al, both to increase yields and to obtain erysolin (25). Specifically, an excess of 1,4-dibromobutane was used to form the single-displacement SN2 product 9 upon addition of potassium phthalimide. The di-substituted product was the only significant side-product and 9 could readily be isolated using standard column chromatography procedures. Displacement of the remaining bromide in 9 was accomplished using a slight excess of sodium thiomethoxide. Trituration and the subsequent removal of residual water afforded 10 in consistently high yields. Deprotection of the phthalimide using hydrazine monohydrate under refluxing conditions, followed by distillation yielded 11 in 80% yield, a significant improvement over previous methods (25). Importantly, it was found that elimination of the acidic workup step and distillation of the oil 11 from the residual solid reaction by-product greatly reduced the net loss of product. Reaction of 11 with an excess of thiophosgene under basic conditions yielded the isothiocyanate 12. Using 12 as a common intermediate, oxidation products 13 and 14 were obtained using either stoichiometric or excess equivalents of m-CPBA. The published procedure that this synthetic effort was based upon reports a yield of 20% over 5 steps for 13 (25). The modification of this procedure as described herein raises the yield to 34% over 5 steps.

Construction of the Isothiocyanate Panel. Utilizing generalized procedures for conversion of a primary amine to an isothiocyanate, a small library of isothiocyanates was constructed. Commercially-available primary amines were selected for inclusion using a number of factors, including steric volume, alkyl ring size, aromaticity, methylene homologation of methylene units, ring substitution patterns, conformational restriction, and bioisosteric substitution. Primary amines were reacted with an excess of thiophosgene and isolated by standard column chromatography (Reaction Scheme 2A). Isothiocyanates were obtained in yields ranging from 9% to quantitative (Reaction Scheme 2B). It was observed that isothiocyanates with low molecular weights and small alkyl chains typically had the lowest yields, likely a result of their increased volatility and loss during purification. Additionally, it is believed that certain functionalities present in the primary amines were not entirely stable to the harsh thiophosgene conditions. Repeated attempts to obtain 22 using thiophosgene resulted in several unidentified breakdown products and a maximum yield of 14%. However, 22 could be obtained in 91% yield employing a different isothiocyanate-installing reagent (26). Substitution of thiophosgene for di-(2-pyridyl)thionocarbonate offered a milder and less toxic means to install isothiocyanates. Although this reagent is much more expensive than thiophosgene, we have observed that its general utility supercedes thiophosgene in nearly all regards (cost being a notable exception). Subjection of 3-picolylamine to di-(2-pyridyl)thionocarbonate readily provided 22.

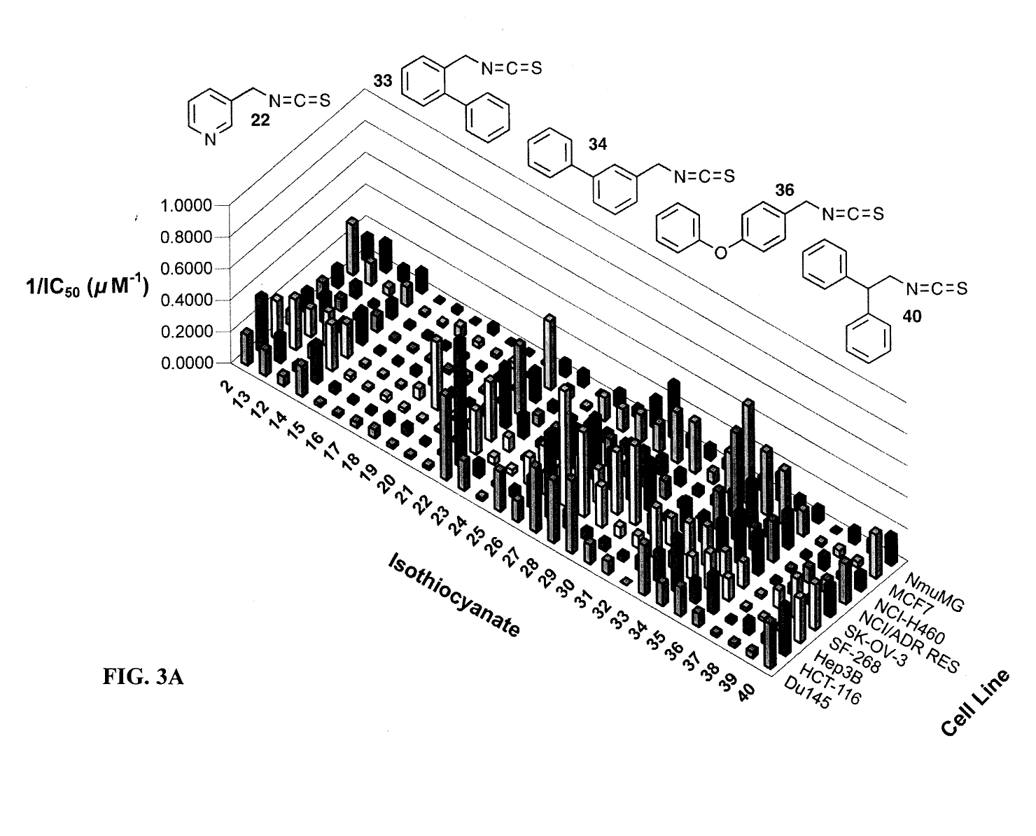

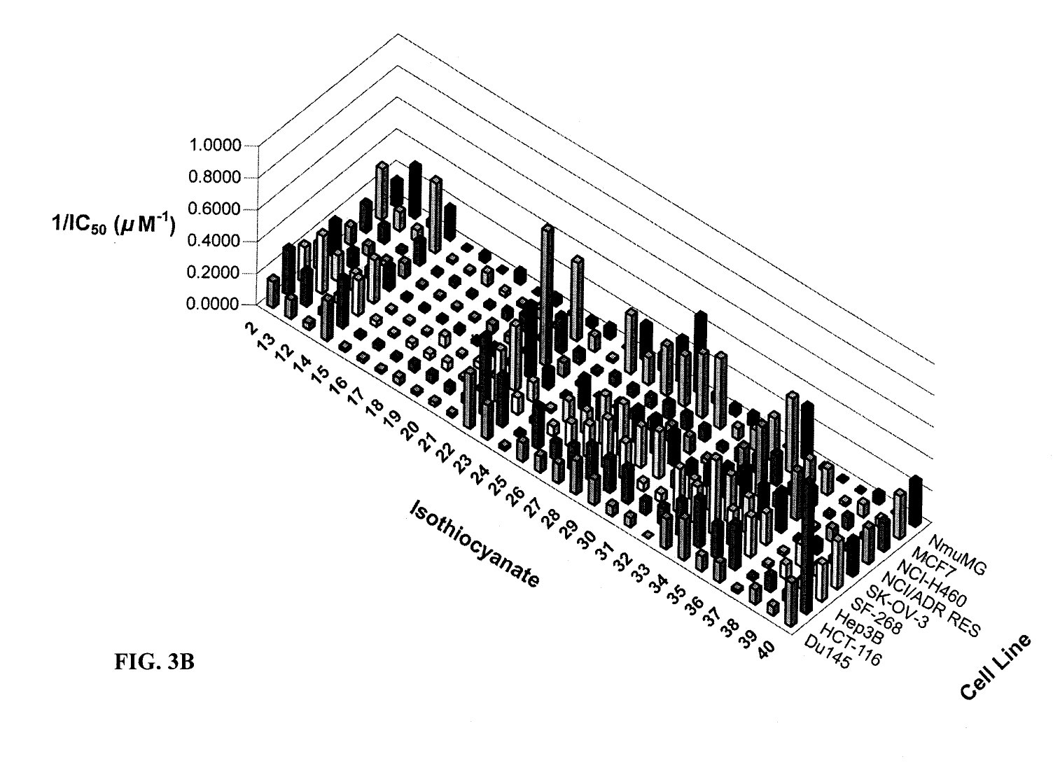

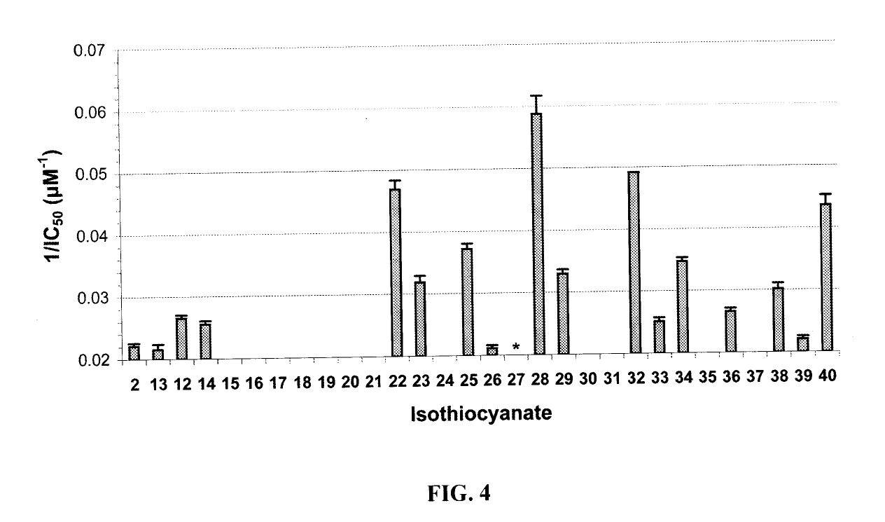

Cytotoxicity of Isothiocyanates. The activity of library members was assessed using three cytotoxicity assays in a total of ten human cancer cell lines representing a broad range of carcinomas, including breast, colon, CNS, liver, lung, ovary, prostate, and a mouse mammary normal epithelial control line (see FIG. 3). The cytotoxicities of L-sulforaphane, D,L-sulforaphane, erucin, and erysolin were also examined. Although the absolute IC₅₀ values obtained using the MTT assay in HT-29 cells are nearly an order of magnitude higher than those obtained using the multiplexed high-throughput assays, the relative IC₅₀ values of ITCs are consistent. However, given the large difference in absolute value, IC₅₀ values obtained using the MTT assay were excluded when calculating average values.

L-Sulforaphane was found to be moderately cytotoxic with an average IC₅₀ of 5.26 μM across all eight human cancer cell lines but was nonspecific because it affected all cells, including NmuMG (IC₅₀=6.59 μM), with similar efficiencies (Table 2). Five compounds were identified from the isothiocyanate library that exhibited overall enhanced activities relative to L-sulforaphane Library members 22 (average IC₅₀=2.54 μM), 33 (average IC₅₀=4.96 μM), 34 (average IC₅₀=3.27 μM), 36 (average IC₅₀=4.84 μM), and 40 (average IC₅₀=4.11 μM) appeared more potent than L-SFN. This was especially evident for 22, which was a highly potent cytotoxin against every human cancer cell line tested (1.18±0.06 μM in the case of NCI/ADR RES, ˜7.5-fold more potent than L-sulforaphane). Additionally, 22 displayed moderate selectivity for cancerous cells over NmuMG (26.04±1.15 μM, ˜4-fold less potent than L-sulforaphane). Taken together these data show that 22 has nearly a 30-fold increase in effectiveness relative to L-sulforaphane. See Table 2

Based on the results of cytotoxicity assays, several trends correlating ITC structure to activity were observed. The absolute stereochemistry and oxidation state of the sulfoxide moiety in L-sulforaphane appear to have an effect on the potency of an ITC-derived cytotoxin. Comparison of average IC₅₀ values for enantiomerically-pure 2 (average IC₅₀=5.26 μM) and racemate 13 (average IC₅₀=7.61 μM) indicate that the L-enantiomer may be more bioactive than the D-enantiomer in specific cell lines. If D-sulforaphane were completely inactive, the racemate 13 would be predicted to have and IC₅₀ 2-fold higher than 2. The results suggest that D-sulforaphane and L-sulforaphane are equally cytotoxic in Du145, HCT-116, Hep3B, SF-268, SK-OV-3, NmuMG, and HT-29 cell lines; their IC₅₀ values lie within standard error of each other. However, notable differences in potency in NCI/ADR RES, NCI-H460, and MCF7 cells suggest that D-sulforaphane is less active in these cell lines (9%, 14%, and 0% bioactive, respectively). The observed cell line specificity for enantiomers could be explained by differences in mechanisms of action of the ITCs in individual cell lines. The level of oxidation of the sulfoxide in 2 also appears to have an effect on bioactivity. Thioether-containing 12 (average IC₅₀=21.99 μM) has significantly higher IC₅₀ values than other sulforaphane-derived analogs while the sulfone 14 (average IC₅₀=5.32 μM) displays similar cytotoxicities on par with 2. While not being tied to any particular biological phenomena or mechanism of action, this trend suggests that generalized oxidation of the sulfur is important for bioactivity, rather than a specific oxidation level. Because effective HDAC inhibitors are sensitive to small differences in the recognition/affinity capping region (27), the observed trend supports the original hypothesis that the 4-(methylsulfinyl)butyl cap group of 3 is non-specific and may be responsible for its relatively modest HDAC inhibition. These observations may have significant ramifications as 13 and 14 are much more amenable to synthesis.

TABLE 6

| | Average IC₅₀ in Cancer | IC₅₀ in NmuMG |
|---|---|---|
| Compound | Cell Lines (μM) | Cell Line (μM) |
| 2 | 5.26 | 6.59 |
| 13 | 7.61 | 3.20 |
| 12 | 21.99^{a} | 23.52 |
| 14 | 5.32 | 5.32 |
| 15 | >50.00^{b} | >200.00 |
| 16 | >50.00 | 26.80 |
| 17 | >50.00^{c} | >200.00 |
| 18 | 29.31^{d} | 14.76 |
| 19 | >50.00 | >200.00 |
| 20 | >50.00^{e} | 182.75 |
| 21 | 35.25^{f} | 65.67 |
| 22 | 2.54 | 26.04 |
| 23 | 9.47 | 15.44 |
| 24 | >50.00 | 150.68 |
| 25 | 10.14 | 4.60 |
| 26 | 10.67 | 9.77 |
| 27 | 6.37 | 3.78 |
| 28 | 6.53 | 2.09 |
| 29 | 5.77 | 16.99 |
| 30 | 9.16 | 21.45 |
| 31 | 17.23 | 15.44 |
| 32 | 8.27^{g} | 16.99 |
| 33 | 4.96 | 180.10 |
| 34 | 3.27 | 3.06 |
| 35 | 7.89 | 65.67 |
| 36 | 4.84 | 186.59 |
| 37 | >50.00 | >200.00 |
| 38 | 11.48 | 17.74 |
| 39 | 18.32 | 13.03 |
| 40 | 4.11 | 3.62 |

^{a}IC₅₀ > 30.00 μM in NCI/ADR RES
^{b}IC₅₀ = 27.55 μM in HCT-116, 32.77 μM in Hep3B
^{c}IC₅₀ = 12.56 μM in MCF7
^{d}Non-inhibitory in NCI/ADR RES
^{e}Calculated solely from Calcein AM data
^{f}Non-inhibitory in Hep3B, NCI/ADR RES, NCI-H460
^{g}Not including data in NCI/ADR RES

Cytotoxicity assays also provided insight regarding the effect of incremental increases in linker length. Library members 19, 23, and 38 differ by the number of methylene groups connecting the isothiocyanate to the recognition/affinity group (phenyl). These three compounds have been studied quite extensively with noticeable differences in bioactivity (28-32). Phenyl isothiocyanate 19 (linker length n=0) was non-inhibitory in nearly all cell lines (average IC₅₀>50.00 μM) while both 23 (n=1, average IC₅₀=9.47 μM) and 38 (n=2, average IC₅₀=11.48 μM) were moderately inhibitory. This trend is supportive of both the published bioactivities of these compounds and what is known about the structure-activity relationships of HDAC inhibitors. The difference of a single methylene in linker length can have a severe impact on the HDAC inhibitory properties of a small molecule as linkers that are too short fail to allow the pharmacophore to access deep within the HDAC cleft. Linkers that are too long do not provide a tight-fitting association of the recognition/affinity group with the enzyme (33). The results presented herein suggest that a minimum of one methylene group adjacent to the isothiocyanate is required to achieve appreciable levels of cytotoxicity.

Many effective HDAC inhibitors contain one or more planar, lipophilic functionalities as part of their recognition/affinity cap group, often as phenyl or phenyl-derived rings, that are thought to participate in critical interactions with the residues near the cleft of the active site (19-24, 33). Comparing the cytotoxicity of compounds 18, 23, 38, and 39 suggests the importance of this planar, aromatic functionality. Structural differences between fully-saturated 18 (average IC₅₀=29.31 μM) and benzyl-derived 23 (average IC₅₀=9.47 μM) suggests that saturation of the benzyl ring corresponds to over a 3-fold increase in potency. A similar effect is observed with the alkene 39 (average IC₅₀=18.32 μM) and 38 (average IC₅₀=11.48 μM). This effect appears to be partially cumulative, as the multiple phenyl rings of compounds 33 (average IC₅₀=4.96 μM), 34 (average IC₅₀=3.27 μM), 35 (average IC₅₀=7.89 μM), 36 (average IC₅₀=4.84 μM), and 40 (average IC₅₀=4.11 μM) promote approximately another 2-fold increase in potency. However, as seen with structural isomers 33, 34, and 35, the connectivity of phenyl rings appears to also have an effect on the relative potency.

Interestingly, it was observed that the absolute stereochemistry of chiral compounds affects the potency of these cytotoxins. Compounds 31 and 32 can be regarded as enantiomers of a conformationally-restricted 23 and show differential IC₅₀ values compared to 23 and to each other. In general, R-enantiomer 32 was approximately 2.0-fold more potent than the S-enantiomer 31 and 1.2-fold more potent than 23. This effect was especially pronounced in Hep3B cells with the IC₅₀s of 9.70±0.37 μM (23), 20.09±0.53 μM (31), and 3.59±0.16 μM (32). This suggests that 32 may more-closely resemble the bioactive conformation of 23; the increased potency of 32 may be attributed to the decrease in the entropy of free rotation upon interaction with its biological target (34). Moreover, this implies that the mechanism or mechanisms by which these compounds exert their cytotoxic behavior is able to differentiate between enantiomers, signifying some level of 3D-scaffolding at the point of interaction.

Compound 22 warrants special discussion as the single most potent compound tested in this panel. In every human cell line, 22 was found to be as active or more so than 2 with an average IC₅₀ of 2.54 μM. Moreover, these effects can likely be attributed solely to substitution of the phenyl in 23 (IC₅₀=11.48±0.77 NCI/ADR RES cells) for the 3-pyridine (IC₅₀=1.18±0.06 μM, NCI/ADR RES cells), a change that leads to as much as a 10-fold increase in potency in NCI/ADR RES cells. If ITCs are indeed precursors to HDAC inhibitors, the cysteine-conjugate of 22 would bear significant resemblance to the HDAC inhibitor pyroxamide 7, especially in respect to their similar 3-pyridine-derived recognition/affinity cap groups. Compound 7, currently in Phase I clinical trials, has been reported to have IC₅₀ values in the 1-20 μM range in several cell lines. In HCT-116 cells, 7 (IC₅₀=6.5±0.5 μM) shows comparable levels of cytotoxicity to 22 (IC₅₀=2.04±0.21 μM) (27, 35). Although the cytotoxicity of 22 is likely due to a combination of cellular mechanisms, including HDAC inhibition, it certainly implicates the importance of the pyridine nitrogen which may participate in crucial interactions with the HDAC active site rim. More importantly, compounds with potencies equal to or greater than 22 may provide another route by which to achieve the HDAC inhibition exhibited by 7.

In contrast, it is noteworthy to address compounds 33 and 36 for their enhanced selectivity toward cancerous cells over healthy cells (Table 2). Even though compounds 33 and 36 both exhibit potencies against cancer cells comparable to 2, they are significantly more selective for cancer cells over normal ones. While compound 2 is moderately selective with only 1.3 times increased specificity for cancer cells over NmuMG cells, 33 and 36 are 36.3- and 38.6-times more selective, respectively. This level of specificity far surpasses selectivities exhibited by other members of the isothiocyanate library. This selectivity is highly dependent on the precise structure of the parent ITC. Comparison of 33, 34, and 35 indicates that cell selectivity is only observed for 2-phenyl 33 (36.3-fold) and 4-phenyl 36 (8.3-fold); 3-phenyl 34 is non-selective.

A small library of ITCs has been synthesized and identified. Several ITC compounds from this library exhibit increased cytotoxicity and selectivity over L-sulforaphane in nine human cancer cell lines. Observations made correlating oxidation state, linker length, lipophilicity, and the stereochemistry of ITCs to their cytotoxicity fall in line with well-established trends found amongst known HDAC inhibitors. These results further indicate the capability of ITCs to act as precursors of HDAC inhibitors and further indicate the utility for synthetic ITCs disclosed herein to be used as dietary chemoprotectants to prevent neoplastic cell growth.

The small library of ITCs may also be administered in an animal subject, whether human or non-human animal in its prodrug form as a glucosinolate analog, such as a glucoraphanin analog. This glucosinolate analog yields a corresponding in vivo metabolite, i.e., an ITC compound. The ITC exhibits chemopreventive/chemotherapeutic activity against neoplastic cell types.

A general methodology for converting a glucoraphanin analog to a sulforaphane derived HDAC inhibitor is depicted in FIG. 1. These metabolites resulting from the glucosinolate compounds may be formed based on myrosinase catalyzed deglycosylation and subsequent Lossen's Rearrangement, as shown below in Reaction Scheme 3:

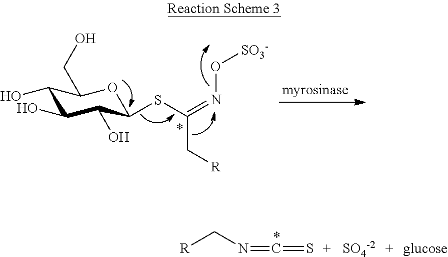

Here, the glucosinolate analog may have various substituent groups attached in place of the glucose moiety such that the glucose moiety is replaced by other sugars that are available in various glycorandomization libraries, such as that of Jon Thorson, of University of Wisconsin, USA. The R substituent may also be a natural or synthetic generic aglycone (which are commercially available). In an exemplary embodiment, the glucosinolate prodrug may be prepared as follows:

Crucial to the generation of new, myrosinase-activated HDAC inhibitors is the ability to equip inhibitor “cap” groups with the thioglucoside and O-sulfated anomeric thiohydroximate functionalities characteristic of Glucoraphanin (1). Myrosinase can effect thiosaccharide hydrolysis leading to Lossen rearrangement (shown in abbreviated form in Scheme 3). Numerous syntheses of glucosinolates have been published; the majority rely on the coupling of commercially available 2,3,4,6-tetra-O-acetyl-1-thio-β-D-gluocopyranose (45) with hydroximoyl chlorides. Thus, the retrosynthesis depicted in Scheme 4 may be used:

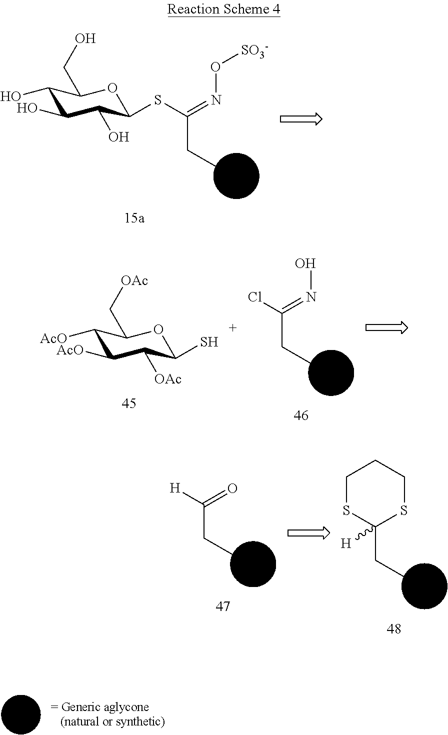

Pivotal to these efforts is generating the hydroximoyl chlorides such as 46 from the primary amines. Aldehydes of the form 47 are easily attainable from corresponding alcohols (via Swern oxidation or Dess-Martin periodinane methods). The S-bearing carbon of the glucosinolate undergoes incorporation within the isothiocyanate moiety by virtue of myrosinase-promoted Lossen rearrangement. To date, it is unclear that myrosinase is involved in Lossen rearrangement other than to provide the starting material for the reaction. There are reports that once deglycosylated, the aglycone undergoes spontaneous Lossen rearrangement. Because of this mechanistic feature, members of the primary amine library initially used to identify ITCs of interest all lack one carbon unit for direct glucosinolate generation. The “missing” carbon is inserted by alkylation of primary amine-derived alkyl bromides with dithiane carbanion so as to afford one carbon homologated species 48. Where appropriate, primary amines of interest can be generated by the corresponding alkyl halide using any one of an assortment of conditions. Of particular interest are conditions shown in Scheme 5 wherein a highly reactive and transient diazonium ion is formed in situ leading to displacement by bromide ion to render 49, 39, and 40. Alternatively, a much milder method of amine halide interconversion may be used.

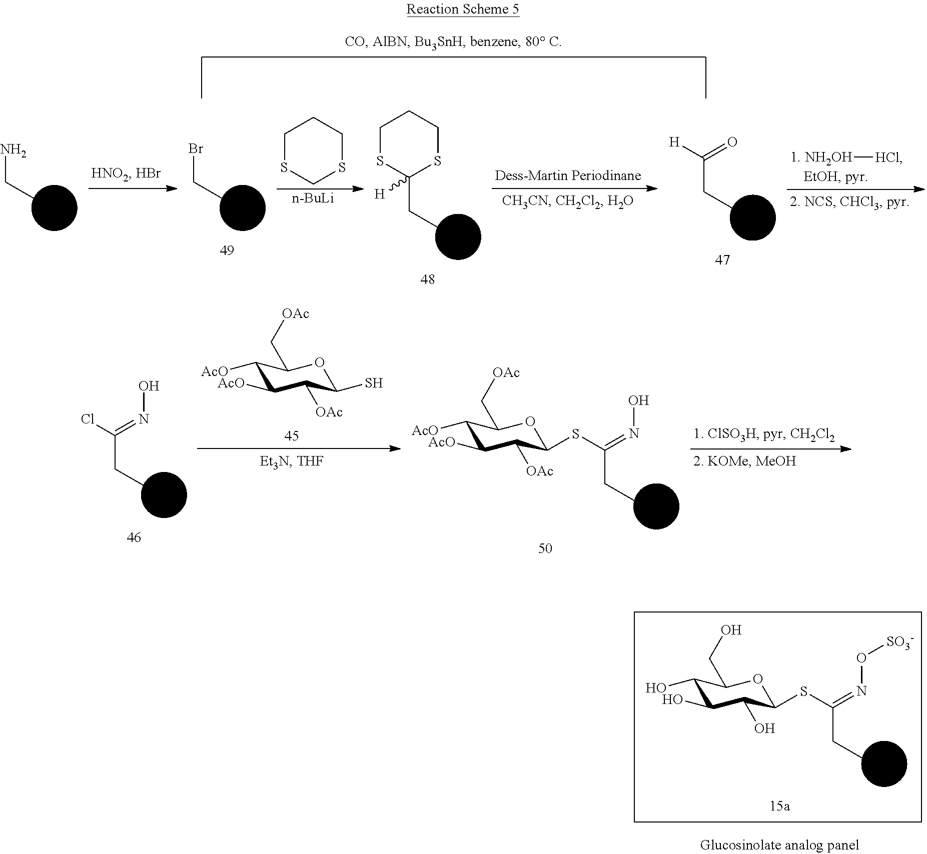

With the alkyl halides in hand, dual one-carbon homologation and aldehyde installation is accomplished in two highly efficient ways. AIBN-induced radical carbonylation of 49 affords, in one step, the aldehyde 47 Yields for such conversions are fair to good although a notable restriction is that benzylic halides do not appear to be sufficiently trapped (following radical formation) by CO, providing instead the fully saturated products. Again, aldehydes such as 47 may be more directly attainable from commercially available alcohols via well known and simple oxidation procedures. Alternatively, the alkylation of alkyl halides with dithiane anion to afford compounds such as 48 is well known as is unmasking of such substances to the corresponding aldehyde. (42, 43) Various examples are also available for bromide displacement with dithiane anion. With aldehydes such as 47, hydroximoyl chlorides of the form 46 may be generated using the well established sequence of oxime formation and N-chlorosuccinimide (NCS)-mediated chlorination (37, 44). Recent applications of this sequence of oxime formation followed by chlorination include work by Prato, Wexler and co-workers (44). Other examples of aldehyde-to-oximyl chloride conversions using Scheme 5 conditions are also available (44) The convergence of chloroximes with commercially available 2,3,4,6-tetra-O-acetyl-1-thio-β-D-gluocopyranose followed by the sequence of sulfation and deacetylation is extremely well established and has played a tremendous role in experiments to elucidate the structural determinants of myrosinase activity (38, 45). The sequence of sulfation and deacetylation depicted in Scheme 5 relies on chemistry developed by Botting and co-workers. Although almost all protocols for sulfation found in the literature are very similar, slight variations in the deacetylation procedure have been noted. Milder variations of Botting's glucosinolate deacylation procedure can be found (45). Crucial among such efforts has been the finding that, while myrosinase is extremely dependent upon the thiosaccharide structure, it is highly tolerant of different aglycone structures (38). This confirms chemical suspicions based on the diversity of natural product glucosinolates which all have the same thiosugar unit yet differ radically in aglycone structure.

The ability of myrosinase (commercially available from Sigma—product T-4528) to convert the glucosinolates described herein to their corresponding ITCs can be determined. These assays employ reaction conditions previously described by Botting and co-workers and the products of reaction are analyzed by reverse phase HPLC using ITCs as standards by which to detect myrosinase-promoted ITC formation.

A panel of reactions may be conducted in the presence and absence of myrosinase wherein HDAC inhibition is assessed. Myrosinase MYR1 from Brassica napus has been expressed in Saccharomyces cerevisiae, but there are no reports of similar experiments in human tissue culture (46). Myrosinase is an extraordinarily robust enzyme. The remarkable stability of myrosinase, even in tissue culture, has allowed elegant studies to evaluate the in vitro cytotoxicity of a number of glucosinolate-derived metabolites against an array of human cancer cell types (47). Based on these considerations and existing precedent, the cellular assays with slight modification may be performed. This approach is shaped in large part by the work of Palmieri and co-workers (47). Thus 22.5 U myrosinase are added to 1 mL of fetal bovine serum containing increasing concentrations of glucosinolates to which ˜2×10⁶ HCT116 cells are added. Cells are then seeded 24 h before transfection into 60-mm culture dishes and transfections are performed as previously described.

### REFERENCES

1. Steinmetz, K. A., Potter, J. D. (1991) “Vegetables, fruit, and cancer. I. Epidemiology,” Cancer Causes Control 2, 325-357.
2. Block, G., Patterson, B., Subar, A. (1992) “Fruit, vegetables, and cancer prevention: A review of the epidemiological evidence,” Nutr. Cancer 18, 1-29.
3. van Poppel, G., Verhoeven, D. T., Verhagen, H., Goldbohm, R. A. (1999) “Brassica vegetables and cancer prevention. Epidemiology and mechanisms,” Adv. Exp. Med. Biol. 472, 159-168.
4. Fenwick, G. R., Heaney, R. K., Mullin, W. J. (1983) “Glucosinolates and their breakdown products in food and food plants,” CRC Crit. Rev. Food Sci. Nutr. 18, 123-201.
5. Tseng, E., Scott-Ramsay, E. A., Morris, M. E. (2004) “Dietary organic isothiocyanates are cytotoxic in human breast cancer MCF-7 and mammary epithelial MCF-12A cell lines,” Exp. Biol. Med. 229, 835-842.
6. Fimognari, C., Nüsse, M., Rossano, C., Iori, R., Cantelli-Forti, G., Hrelia, P. (2002) “Growth inhibition, cell-cycle arrest and apoptosis in human T-cell leukemia by the isothiocyanate sulforaphane,” Carcinogenesis. 23, 581-586.
7. Zhang, Y., Talalay, P., Cho, C. G., Posner, G. H. (1992) “A major inducer of anticarcinogenic protective enzymes from broccoli: Isolation and elucidation of structure,” Proc. Natl. Acad. Sci. U.S.A. 89, 2399-2403.
8. Zheng, G. Q., Kenney, P. M., Lam, K. T. (1992) “Phenylalkyl isothiocyanate-cysteine conjugates as glutathione S-transferase stimulating agents,” J. Med. Chem. 35, 185-188.
9. Morimitsu, Y., Nakagawa, Y., Hayashi, K., Fujii, H., Kumagai, T., Nakamura, Y., Osawa, T., Horio, F., Itoh, K., Iida, K., Yamamoto, M., Uchida, K. (2002) “A sulforaphane analogue that potently activates the Nrf2-dependent detoxification pathway,” J. Biol. Chem. 277, 3456-3463.
10. Basten, G. P., Bao, Y., Williamson, G. (2002) “Sulforaphane and its glutathione conjugate but not sulforaphane nitrile induce UDP-glucuronosyl transferase (UGT1A1) and glutathione transferase (GSTA1) in cultured cells,” Carcinogenesis. 23, 1399-1404.
11. Zhang, Y., Kensler, T. W., Cho, C., Posner, G. H., Talalay, P. (1994) “Anticarcinogenic activities of sulforaphane and structurally related synthetic norbornyl isothiocyanates,” Proc. Natl. Acad. Sci. U.S.A. 91, 3147-3150.
12. Hu, R., Hebbar, V., Kim, B.-R., Chen, C., Winnik, B., Buckley, B., Soteropoulos, P., Tolias, P., Hart, R. P., Kong, A.-N. T. (2004) “In vivo pharmacokinetics and regulation of gene expression profiles by isothiocyanate sulforaphane in the rat,” J. Pharm. Exp. Ther. 310, 263-271
13. Bonnesen, C., Eggleston, I. M., Hayes, J. D. (2001) “Dietary indoles and isothiocyanates that are generated from cruciferous vegetables can both stimulate apoptosis and confer protection against DNA damage in human colon cell lines,” Cancer Res. 61, 6120-6130.
14. Jackson, S. J. T., Singletary, K. W. (2004) “Sulforaphane: a naturally occurring mammary carcinoma mitotic inhibitor which disrupts tubulin polymerization,” Carcinogenesis. 25, 219-227.
15. Kassahun, K., Davis, M., Hu, P., Martin, B., Baillie, T. (1997) “Biotransformation of the naturally occurring isothiocyanate sulforaphane in the rat: Identification of phase I metabolites and glutathione conjugates,” Chem. Res. Toxicol. 10, 1228-1233.
16. Myzak, M. C., Dashwood, R. H. (2006) “Chemoprotection by sulforaphane: Keep one eye beyond Keap1,” Can. Let. 233, 208-218.
17. Myzak, M. C., Karplus, P. A., Chung, F., Dashwood, R. H. (2004) “A novel mechanism of chemoprotection by sulforaphane: Inhibition of Histone Deacetylase,” Cancer Res. 64, 5767-5774.
18. Marks, P. A., Richon, V. M., Breslow, R., Rifkind, R. A. (2001) “Histone deacetylase inhibitors as new cancer drugs,” Curr. Op. Oncol. 13, 477-483.
19. Furumai, R., Komatsu, Y., Nishino, N., Khochbin, S., Yoshida, M., Horinuchi, S. (2001) “Potent histone deacetylase inhibitors built from trichostatin A and cyclic tetrapeptide antibiotics including trapoxin,” Proc. Natl. Acad. Sci. USA. 98, 87-92.
20. Jung, M., Brosch, G., Kolle, D., Scherf, H., Gerhauser, C., Loidl, P. (1999) “Amide analogs of trichostatin as inhibitors of histone deacetylase and inducers of terminal cell differentiation,” J. Med. Chem. 42, 4669-4679.
21. Lavoie, R., Bouchain, D., Frechette, S., Woo, S., khalil, E. A., Leit, S., Fournl, M., Yan, P. T., Trachy-Bourget, M., Beaulieu, C., Li, z., Besterman, J., Delorme, D. (2001) “Design and synthesis of a novel class of histone deacetylase inhibitors,” Bioorg. Med. Chem. Lett. 11, 2847-2850.
22. Wittich, S., Scherf, H., Xie, C., Brosch, G., Loidl, P., Gerhauser, C., Jung, M. (2002) “Structure-activity relationship of phenylalanine-containing inhibitors of histone deacetylase: In vitro enzyme inhibition, induction of differentiation, and inhibition of proliferation in friend leukemic cells,” J. Med. Chem. 45, 3296-3309.
23. Taunton, J., Hassig, C. A., Schreiber, S. L. (1996) “A mammalian histone deacetylase related to the yeast transcriptional regulator rpd3p,” Science. 272, 408-411.
24. Chung, F., Jiao, D., Conaway, C. C., Smith, T. J., Yang, C. S., Yu, M. C. (1997) “Chemopreventive potential of thiol conjugates of isothiocyanates for lung cancer and a urinary biomarker of dietary isothiocyanates,” J. Cell. Biochem. 27, 76-85.
25. Vermeulen, M., Zwanenburg, B., Chittenden, G. J. F., Verhagen, H. (2003) “Synthesis of isothiocyanate-derived mercapturic acids,” Eur. J. Med. Chem. 38, 729-737.
26. Park, S., Hayes, B. L., Marankan, F., Mulhearn, D. C., Wanna, L., Mesecar, A. D., Santarseiro, B. D., Johnson, M. E., Venton, D. L. (2003) “Regioselective covalent modification of hemoglobin in search of antisickling agents,” J. Med. Chem. 46, 936-953.
27. Remiszewski, S. W., Sambucetti, L. C., Atadja, P., Bair, K. W., Cornell, W. D., Green, M. A., Howell, K. L., Jung, M., Kwon, P., Trogani, N, Walker, H. (2002) “Inhibitors of human histone deacetylase: Synthesis and enzyme and cellular activity of straight chain hydroxamates,” J. Med. Chem. 45, 753-757.
28. Manesh, C., Kuttan, G. (2003) “Anti-tumour and anti-oxidant activity of naturally occurring isothiocyanates,” J. Exp. Clin. Can. Res. 22, 193-199.
29. Wattenberg, L. W. (1977) “Inhibition of carcinogenic effects of polycyclic hydrocarbons by benzyl isothiocyanate and related compounds,” J. Nat. Can. Inst. 58, 395-398.
30. Nakamura, Y. (2004) “Cancer chemoprevention by isothiocyanates: Cancerous cell-specific control of cell proliferation and its molecular mechanism,” Kankyo Hen'igen Kenkyu 26, 253-258.
31. Akagi, K., Masashi, S., Ogawa, K., Hirose, M., Goshima, H., Shirai, T. (2003) “Involvement of toxicity as an early event in urinary bladder carcinogenesis induced by phenethyl isothiocyanate, benzyl isothiocyanate, and analogues of F344 rats,” Toxicologic Path. 31, 388-396.
32. Tang, L., Zhang, Y. (2005) “Mitochondria are the primary target in isothiocyanate-induced apoptosis in human bladder cancer cells,” Mol. Cancer. Ther. 4, 1250-1259.
33. Miller, T. A., Witter, D. J., Belvedere, S. (2003) “Histone deacetylase inhibitors,” J. Med. Chem. 46, 5097-5116.
34. Khan, A. R., Parrish, J. C., Fraser, M. E., Smith, W. W., Bartlett, P. A., James, M. N. G. (1998) “Lowering the entropic barrier for binding conformationally flexible inhibitors to enzymes,” Biochem. 37, 16839-16845.
35. Kutko, M. C., Glick, R. D., Butler, L. M., Coffey, D. C., Rifkind, R. A., Marks, P. A., Richon, V. M., LaQuaglia, M. P. (2003) “Histone deacetylase inhibitors induce growth suppression and cell death in human rhabdomyosarcoma in vitro,” Clin. Can. Res. 9, 5749-5755.
36. Cottaz, S.; Henrissat, B.; Driguez, H. (1996) “Mechanism-based Inhibition and Stereochemistry of Glucosinolate Hydrolysis by Myrosinase,” Biochemistry 35, 15256-15259.
37. Davidson, N. E.; Rutherford, T. J.; Botting, N. P. (2001) “Synthesis, analysis and rearrangement of novel unnatural glucosinolates,” Carbohydrate Res. 330, 295-307.
38. Botti, M. G.; Taylor, M. G.; Botting, N. P. (1995) “Studies on the Mechanism of Myrosinase,” J. Biol. Chem. 270, 20530-20535.
39. (a) Volkmann, R. A.; Kelbaugh, P. R.; Nason, D. M.; Jasys, V. J. (1993) “2-Thioalkyl Penems: An Efficient Synthesis of Sulopenem, a (5R,6S)-6-(1(R)-Hydroxyethyl)-2-[(cis-1-oxo-3-thiolanyl)thio]-2-penem Antibacterial,” J. Org. Chem. 1992, 57, 4352-4361; (b) Dener, J. M.; Zhang, L.-H.; Rapoport, H. “An Effective Chirospecific Synthesis of (+)-Pilocarpine from L-Aspartic Acid” J. Org. Chem. 58, 1159-1166.
40. Katritzky, A. R.; Al-Omran, F.; Patel, R. C.; Thind, S. S. (1980) “Improved Methods for Conversion of Primary Amines into Bromides,” Perkin Trans 1 1890-1894.
41. Ryu, I.; Kusano, K.; Ogawa, A.; Kambe, N.; Sonada, N. (1990) “Free-Radical Carbonylation. Efficient Trapping of Carbon Monoxide by Carbon Radicals,” J. Am. Chem. Soc. 112, 1295-1297.
42. (a) Grigg, R.; Markandu, J.; Surendrakumar, S.; Thornton-Pett, M.; Warnock, W. J. (1992) “X═Y—ZH Systems as Potential 1,3-Dipoles. Part 37 Generation of Nitrones from Oximes. Tandem Intramolecular 1,3-AzaprotioCyclotransfer-Intramolecular 1,3-Dipolar Cycloaddition Reactions. Class 4 Processes,” Tetrahedron 48, 10399-10422; (b) Adams, T. C.; Dupont, A. C.; Carter, J. P.; Kachur, J. F.; Guzewska, M. E.; Rzeszotarski, W. J.; Farmer, S. G.; Noronha-Blob, L.; Kaiser, C. (1991) “Aminoalkynyldithianes. A New Class of Calcium Channel Blockers” J. Med. Chem. 34, 1585-1593.
43. Dithiane cleavage to afford aldehyde: Langille, N. F.; Dakin, L. A.; Panek, J. S. (2003) “A Mild, Chemoselective Protocol for the Removal of Thioketals and Thioacetals Mediated by Dess-Martin Periodinane” Org. Lett. 5, 575-578.
44. (a) DaRos, T.; Prato, M.; Lucchini, V. (2000) “Additions of Azomethine Ylides to Fullerene C60 Assisted by a Removable Anchor” J. Org. Chem. 65, 4289-4297; (b) Lam, P. Y. S.; Adams, J. J.; Clark, C. G.; Calhoun, W. J.; Luettgen, J. M.; Knabb, R. M.; Wexler, R. R. (2003) “Discovery of 3-Amino-4-Chlorophenyl P1 as a Novel and Potent Benzamidine Mimic Via Solid-Phase Synthesis of an Isoxazoline Library” Bioorg. Med. Chem. Lett. 13, 1795-1799.
45. (a) Lefoix, M.; Tatibouet, A.; Cottaz, S.; Driguez, H.; Rollin, P. (2002) “Carba-glucotropaeolin: the first non-hydrolyzable glucosinolate analogue to inhibit myrosinase” Tet. Lett. 43, 2889-2890; (b) Cassel, S.; Casenave, B.; Deleris, G.; Latxangue, L.; Rollin, P. (1998) “Exploring an Alternative Approach to the Synthesis of Arylalkyl and Indolylmethyl Glucosinolates” Tetrahedron 54, 8515-8524; (c) Mavratzotis, M.; Dourtoglou, V.; Lorin, C.; Rollin, P. (1996) “Glucosinolate Chemistry. First Synthesis of Glucosinolates Bearing an External Thio-Function” Tet. Lett. 37, 5699-5700
46. Chen, S.; Halkier, B. A. (1999) “Functional Expression and Characterization of the Myrosinase MYR1 from Brassica napus in Saccharomyces cerevisiae” Protein Expression and Purification 17, 414-420.
47. Nastruzzi, C.; Cortesi, R.; Esposito, E.; Menegatti, E.; Leoni, O.; Iori, R.; Palmieri, S. (1996) “In Vitro Cytotoxic Activity of Some Glucosinolate-derived Products Generated by Myrosinase Hydrolysis” J. Agric. Food Chem. 44, 1014-1021.

What is claimed is:

1. Compounds of Formula I:
R—N═C═S
wherein R is selected from the group consisting of dimethylpropyl, C₃-C₁₀ mono- or bicycloalkyl, C₆-C₁₀ mono- or bicycloakenyl, halobenzyl, alkyloxybenzyl, tetrahydronaphthalenyl, biphenyl-C₁-C₆-alkyl, phenoxybenzyl-C₁-C₆-alkyl, and pyridinyl-C₁-C₆-alkyl;
N-acetyl cysteine conjugates thereof;
and salts thereof.

2. Compounds of claim 1, wherein R is selected from the group consisting of:

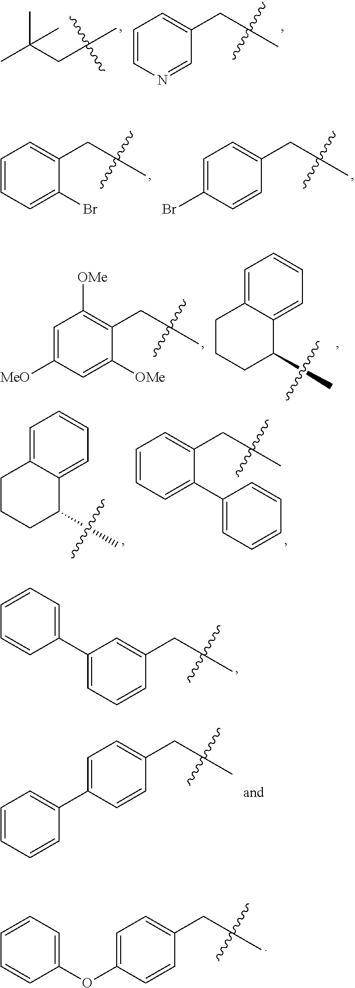

3. Compounds of claim 1, wherein R is selected from the group consisting of:

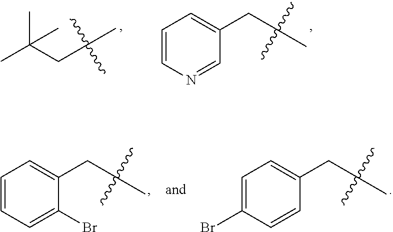

4. Compounds of claim 1, wherein R is selected from the group consisting of:

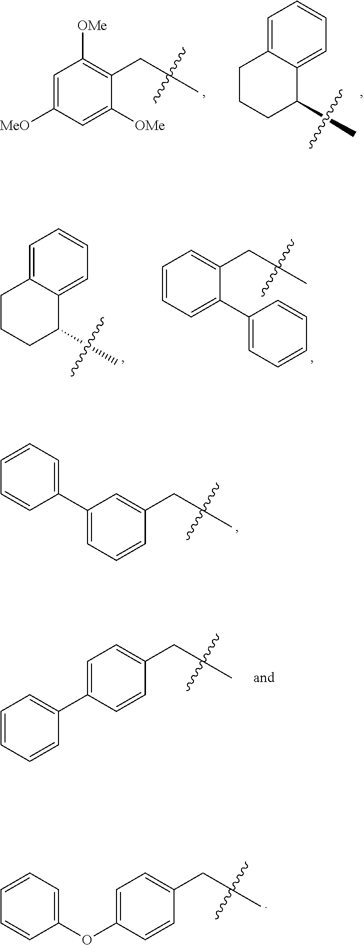

5. A pharmaceutical composition for inhibiting neoplastic cell growth comprising one or more compounds of claim 1, or pharmaceutically suitable salts thereof, in combination with a pharmaceutically-suitable carrier.

6. The pharmaceutical composition of claim 5, wherein the carrier is a solid carrier.

7. The pharmaceutical composition of claim 5, wherein the carrier is a liquid carrier.

8. A method of inhibiting growth of cancer cells comprising treating the cancer cells with an effective growth-inhibiting amount of one or more compounds of claim 1, or pharmaceutically suitable salts thereof.

9. The method of claim 8, wherein an amount of one or more of the compounds is administered to a human cancer patient in need thereof which is effective to inhibit the growth of the cancer.

10. The method of claim 9, wherein the amount of one or more of the compounds is administered parenterally in combination with a pharmaceutically-acceptable liquid or solid carrier.

11. The method of claim 9, wherein the amount of one or more of the compounds is administered intravenously in combination with a pharmaceutically-acceptable liquid carrier.

12. The method of claim 9, wherein the amount of one or more of the compounds is administered orally in combination with a pharmaceutically-acceptable liquid or solid carrier.

13. Compounds of Formula II: 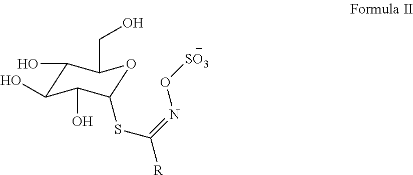
wherein R is selected from the group consisting of: 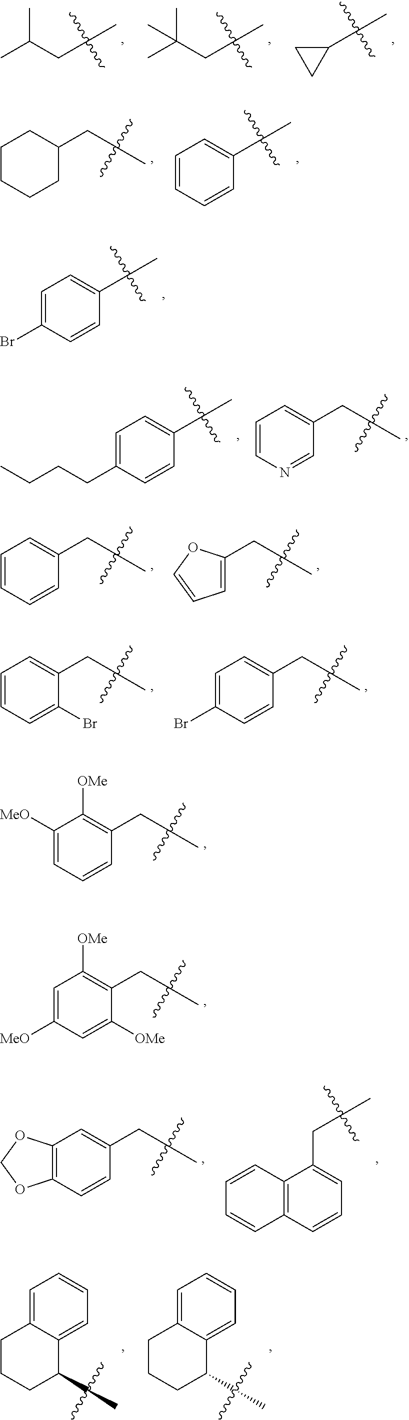 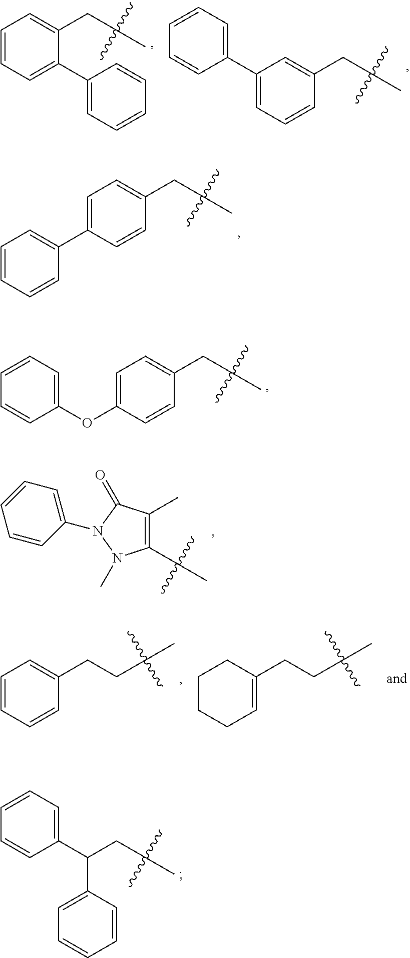
and
N-acetyl cysteine conjugates thereof;
and salts thereof.

14. A pharmaceutical composition for inhibiting neoplastic cell growth comprising one or more compounds of claim 13, or pharmaceutically suitable salts thereof, in combination with a pharmaceutically-suitable carrier.

15. The pharmaceutical composition of claim 14, wherein the carrier is a solid carrier.

16. The pharmaceutical composition of claim 14, wherein the carrier is a liquid carrier.

17. A method of inhibiting growth of cancer cells in mammals comprising:
(a) administering to the mammal a cancer cell growth-inhibiting amount of one or more of compounds of Formula II or a pharmaceutically suitable salt thereof: 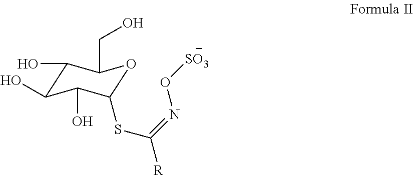
wherein R is selected from the group consisting of: 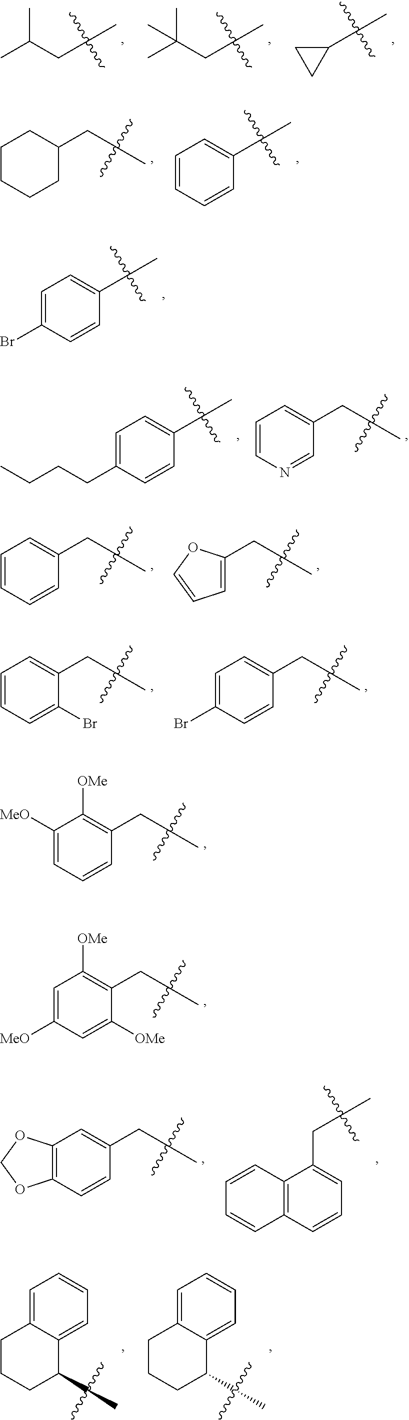 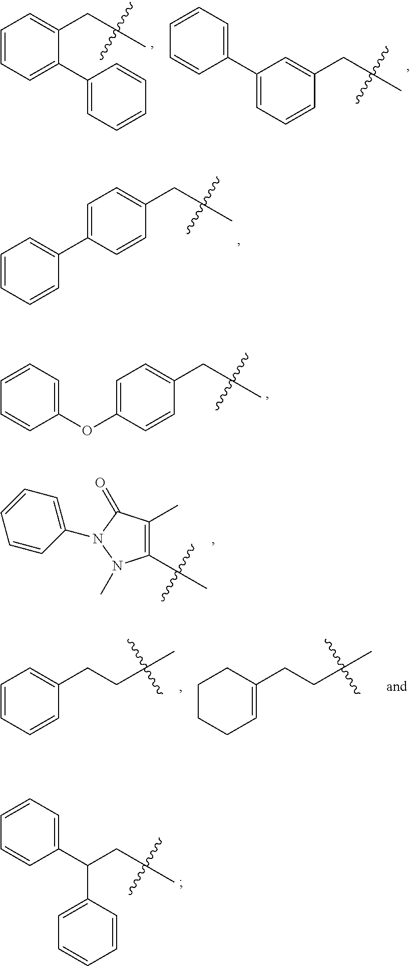
and
(b) wherein the amount of the administered Formula II compound yields an in vivo metabolite in the mammal selected from the group consisting of:

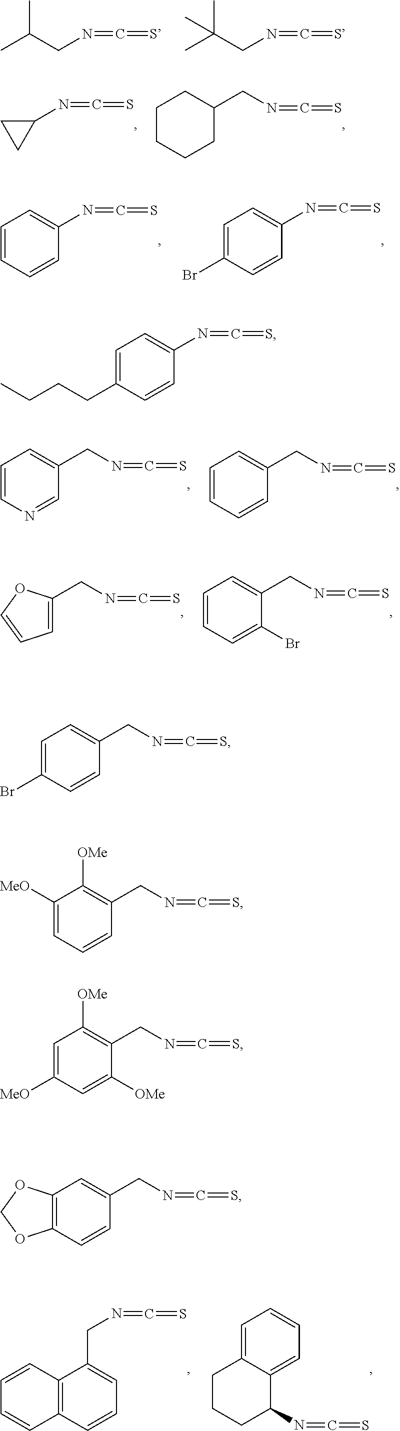

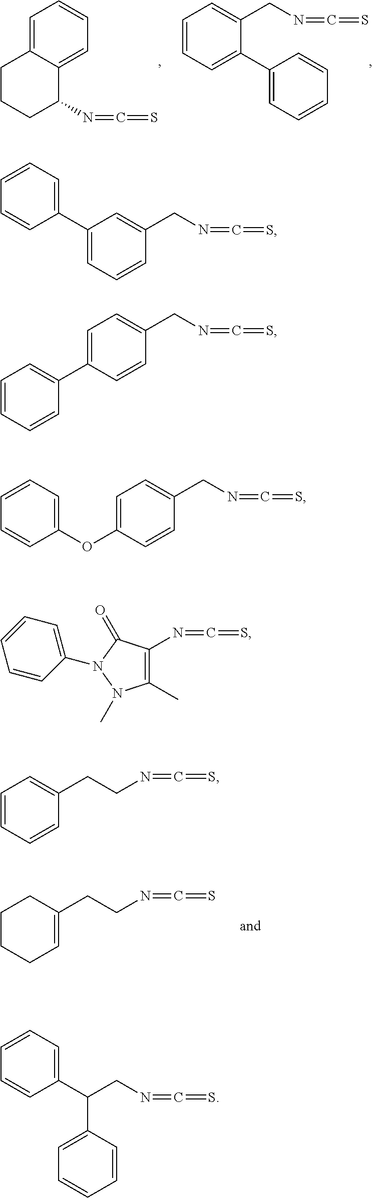

18. The method of claim 17, wherein an amount of one or more of the compounds is administered to a human cancer patient in need thereof which is effective to inhibit the growth of the cancer.

19. The method of claim 17, wherein the amount of one or more of the compounds is administered parenterally in combination with a pharmaceutically-acceptable liquid or solid carrier.

20. The method of claim 17, wherein the amount of one or more of the compounds is administered intravenously in combination with a pharmaceutically-acceptable liquid carrier.

21. The method of claim 17, wherein the amount of one or more of the compounds is administered orally in combination with a pharmaceutically-acceptable liquid or solid carrier.

22. A pharmaceutical composition for inhibiting neoplastic cell growth comprising one or more compounds selected from the group consisting of: 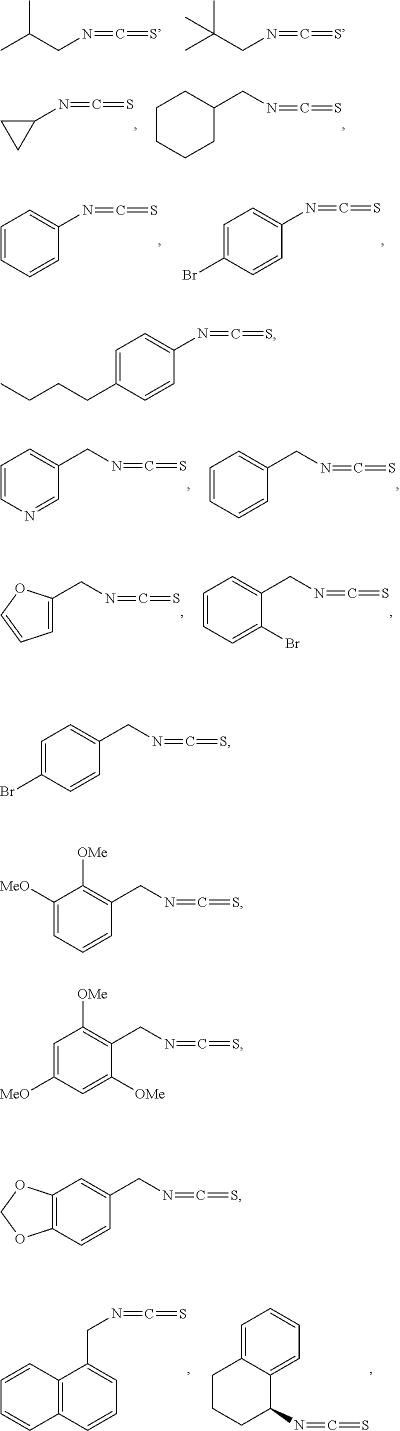 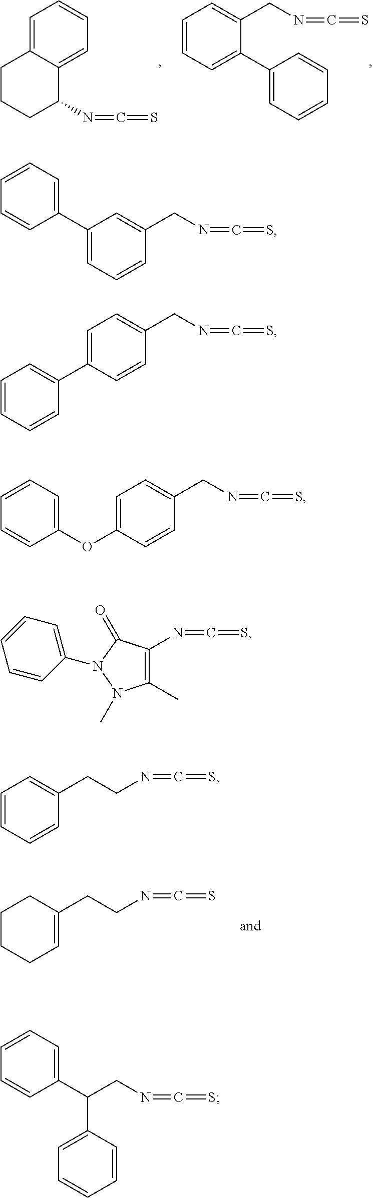
N-acetyl cysteine conjugates thereof;
and pharmaceutically suitable salts thereof;
in combination with a pharmaceutically-suitable carrier.

23. The pharmaceutical composition of claim 22, wherein the carrier is a solid carrier.

24. The pharmaceutical composition of claim 22, wherein the carrier is a liquid carrier.

25. A method of inhibiting growth of cancer cells in mammals comprising:
(a) administering to the mammal a cancer cell growth-inhibiting amount of one or more of compounds selected from the group consisting of: 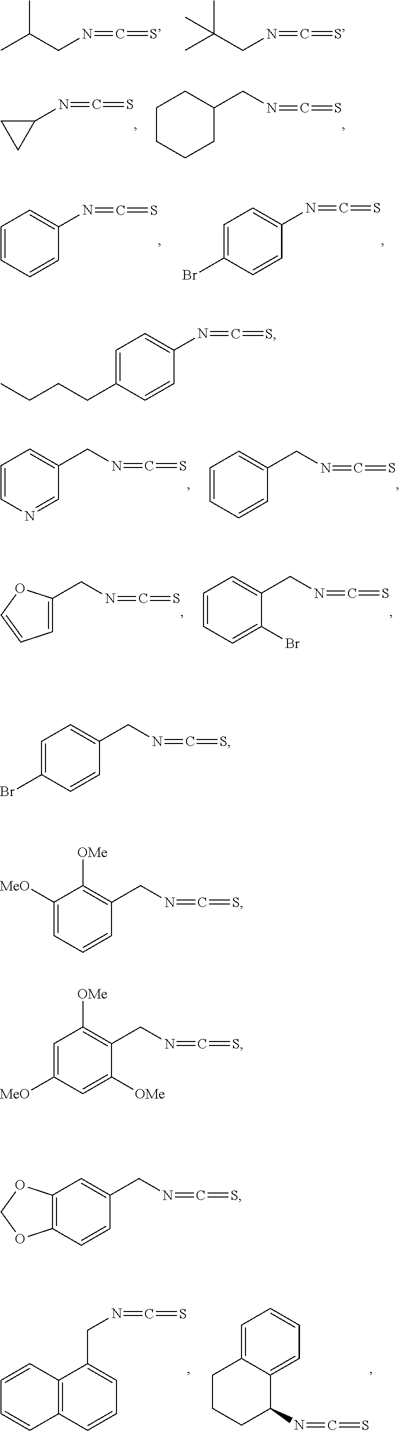 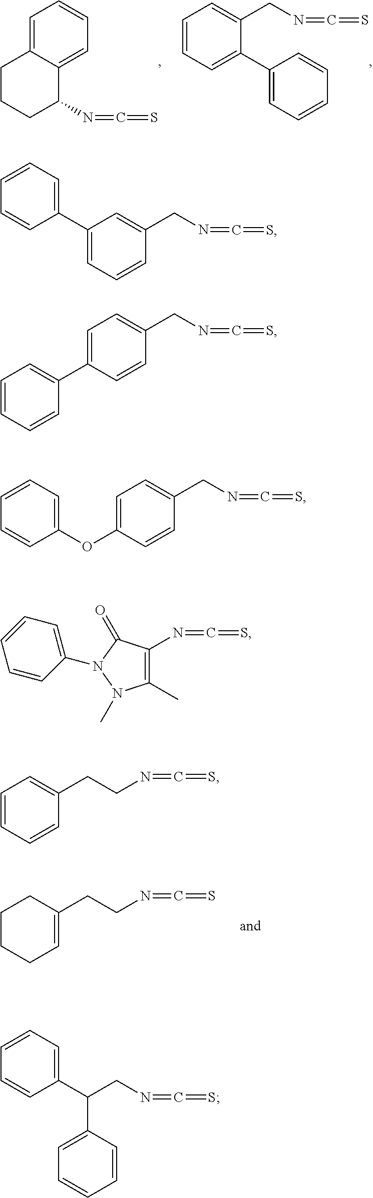
N-acetyl cysteine conjugates thereof;
and pharmaceutically suitable salts thereof.

26. The method of claim 25, wherein an amount of one or more of the compounds is administered to a human cancer patient in need thereof which is effective to inhibit the growth of the cancer.

27. The method of claim 25, wherein the amount of one or more of the compounds is administered parenterally in combination with a pharmaceutically-acceptable liquid or solid carrier.

28. The method of claim 25, wherein the amount of one or more of the compounds is administered intravenously in combination with a pharmaceutically-acceptable liquid carrier.

29. The method of claim 25, wherein the amount of one or more of the compounds is administered orally in combination with a pharmaceutically-acceptable liquid or solid carrier.

30. A method of inhibiting histone deacetylase activity in mammals comprising:
(a) administering to the mammal a histone deacetylase activity-inhibiting amount of one or more of compounds of Formula II or a pharmaceutically suitable salt thereof: 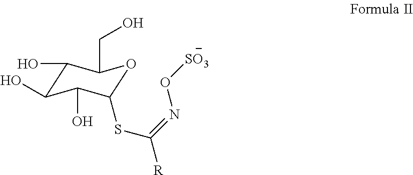
wherein R is selected from the group consisting of: 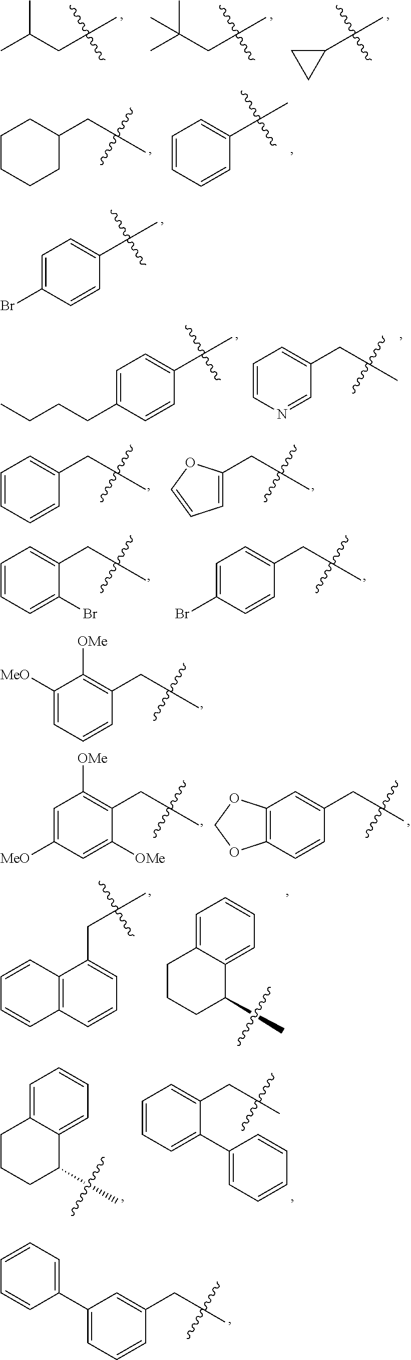 [chemical structure]
